(19) United States
(12) Patent Application Publication
(10) Pub. No.: US 2023/0307141 A1
(43) Pub. Date: Sep. 28, 2023

(54) MODELS FOR ACCURATE PATIENT ACUITY

(71) Applicant: MatrixCare, Inc., Bloomington, MN (US)

(72) Inventors: Vivek KUMAR, Eden Prairie, MN (US); Coleen DANIELSON, Fergus Falls, MN (US); Samsudhin H., Chennai (IN)

(21) Appl. No.: 18/167,289

(22) Filed: Feb. 10, 2023

Related U.S. Application Data
(60) Provisional application No. 63/269,790, filed on Mar. 23, 2022.

(51) Int. Cl. G16H 50/30

(52) U.S. Cl. CPC G16H 50/30

(57) ABSTRACT

Techniques for improved computer modeling are provided. Resident data describing a resident of a healthcare facility is received, and a set of resident attributes corresponding to a defined set of features is extracted from the resident data. An acuity score is generated for the resident by processing the set of resident attributes using an acuity model, the acuity model specifying a respective weight for each respective feature in the defined set of features. One or more interventions are initiated for the resident based on the acuity score.

### CROSS-REFERENCE TO RELATED APPLICATIONS

This application claims priority to U.S. Provisional Patent Application No. 63/269,790, filed Mar. 23, 2022, the entire content of which is incorporated herein by reference in its entirety.

### STATEMENT REGARDING PRIOR DISCLOSURES BY THE INVENTOR OR A JOINT INVENTOR

Beginning in June of 2020, Applicant conducted confidential experiments with multiple healthcare/nursing-home facilities to determine the utility, accuracy, deploy-ability, and suitability of prototype systems and methods that to evaluate patient acuity. As a result of these confidential activities and further development, additional techniques, systems, features and methods were discovered that further enhance an accuracy of a system or method to evaluate acuity, in addition to increasing the operability and efficiency of the same in application.

Beginning in October of 2021, Applicant engaged in certain activities provided to third parties which may constitute a disclosure or offer for sale. These activities occurred less than one year prior to the effective filing date of this application.

### INTRODUCTION

Embodiments of the present disclosure relate to computer modeling. More specifically, embodiments of the present disclosure relate to using computer modeling to evaluate patient acuity.

In conventional care settings, such as residential care facilities (e.g., nursing homes), a wide variety of resident characteristics are assessed and monitored in an effort to reduce or prevent worsening any resident's condition. An acute change of condition may refer to a sudden and clinically important deviation from a patient's (or resident's) baseline in physical, cognitive, behavioral, and/or functional domains. Without intervention, such deviations can lead to clinically significant complications.

Additionally, a significant fraction of residents or patients end up transferring or returning to hospitals shortly after admission to long-term care facilities. Such admissions (or readmissions) amount to billions of dollars spent. Additionally, readmissions can generally affect a variety metrics used to evaluate long-term residential facilities, such as the quality metrics defined by the Centers for Medicare and Medicaid Services. Generally, readmissions shortly after entry to the facility (e.g., within thirty days) are penalized, having an adverse effect on these scores.

Care settings strive to provide higher levels of care in order to improve resident conditions and reduce such readmissions. However, conventional approaches are entirely manual (relying on the expertise of individual caretakers to recognize concerns), and are generally inflexible and inaccurate (failing to identify those most in need of additional care). Further, given the vast amount of data associated with reach resident, it is infeasible for users to manually identify potential concerns.

Improved systems and techniques to automatically evaluate patients are needed.

### SUMMARY

According to one embodiment presented in this disclosure, a method is provided. The method includes: identifying, for a first resident, a first set of resident attributes corresponding to a defined set of features, wherein at least one of the first set of resident attributes is generated using one or more machine learning models; determining that one or more resident attributes of the first set of attributes have changed; and in response to determining that one or more of the first set of resident attributes have changed, generating a first acuity score for the first resident by processing the first set of resident attributes using an acuity model, wherein: the acuity model specifies a respective weight for each respective feature in the defined set of features, and the first acuity score indicates a probability that the first resident will be hospitalized within a defined timeframe.

According to one embodiment presented in this disclosure, a method is provided. The method includes: receiving resident data describing a first resident of a healthcare facility; extracting, from the resident data, a first set of resident attributes corresponding to a defined set of features; generating a first acuity score for the first resident by processing the first set of resident attributes using an acuity model, the acuity model specifying a respective weight for each respective feature in the defined set of features; and initiating one or more interventions for the first resident based on the first acuity score.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example workflow for generating acuity models based on historical data.

FIG. 2 depicts an example workflow for generating acuity scores and interventions using acuity models.

FIG. 3 depicts an example workflow for using machine learning to generate fall risk input for acuity evaluations.

FIG. 4 depicts an example workflow for generating diagnoses input for acuity evaluations.

FIG. 5 depicts an example workflow for generating assistance input for acuity evaluations.

FIG. 6 depicts an example workflow for generating clinical assessment input for acuity evaluations.

FIG. 7 depicts an example workflow for generating therapy input for acuity evaluations.

FIG. 8 depicts an example graphical user interface for acuity evaluations.

FIG. 9 is a flow diagram depicting an example method for generating acuity models.

FIG. 10 is a flow diagram depicting an example method for generating acuity scores using acuity models.

FIG. 11 is a flow diagram depicting an example method for identifying patient attributes to evaluate acuity.

FIG. 12 is a flow diagram depicting an example method for generating and aggregating patient acuity.

FIG. 13 is a flow diagram depicting an example method for generating acuity scores by processing patient attributes using an acuity model.

FIG. 14 is a flow diagram depicting an example method for generating an acuity score using an acuity model.

FIG. 15 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved modeling of user acuity and improved resident care.

In some embodiments, an acuity model can be generated and used as an assessment tool for clinicians to assist in identifying at-risk residents, improving care, and preventing hospital readmissions. In some embodiments, by monitoring for changes in the condition for each resident, the system is able to identify ailing residents and assist with reallocating resources (including staff) to units with higher acuity (which generally require more care, resources, and staff time). In some embodiments, the acuity model enables real-time insights, including alerts that notify the clinician or caretaker when there has been a change in condition with any resident (e.g., within the past 24 hours), and provides the clinician with an actionable assessment and intervention details. In conventional settings, caretakers must manually review each resident's data (often consuming an hour or more per resident) to determine whether any changes in condition occurred (e.g., in the past 24 hours). As each caretaker can be responsible for a significant number of residents during his/her shift, a significant amount of time and resources are wasted performing such reviews. Aspects of the present disclosure can reduce this waste, as well as reducing the risk and/or potential injury to residents.

Embodiments of the present disclosure can generally enable proactive and quality care for residents and dynamic reallocation of resources and staff, and that further helps to prevent or reduce adverse events such as re-hospitalizations, sepsis, or severe changes in condition. This autonomous and continuous updating on changing conditions with respect to individual patients and across groups of patients in a facility enables a wide variety of improved results, including not only improved outcomes for the residents (e.g., reduced re-admission, early identification of declining status, reallocating resources, selecting and applying targeted interventions, and the like) but also improved computational efficiency and accuracy of the evaluation and solution process.

In some embodiments, a variety of historical resident or patient data is collected and evaluated to build one or more acuity models, where the acuity model specifies a set of features (e.g., resident attributes) and a corresponding set of weights for those features. For example, the acuity model may have a first weight for one feature (e.g., a set of defined diagnoses) that is roughly double the weight of another feature (e.g., a set of defined clinical assessments), such that the first feature (diagnoses) has twice the impact of the second. In some embodiments, the acuity model is a statistical model (e.g., with manually curated weights). That is, the features and weights of the acuity model may be determined manually, or using statistical analysis of the historical data. In some embodiments, the acuity model is a machine learning model (e.g., with learned weights). That is, the features and weights of the acuity model may be learned during a training phase. In some aspects, the acuity model is a static model that, once generated, remains fixed. In other embodiments, the acuity model may be updated (either periodically or upon specified criteria). That is, the acuity model may be updated based on renewed statistical analysis, based on a new training or refinement phase, and the like.

In embodiments, the acuity model can generally be used to generate an acuity score or acuity classification for a given resident based on their attributes. For example, the system may determine the resident's fall risk (e.g., generated using a machine learning model), diagnoses, needed point-of-care or assistance actions, clinical assessments, therapies (e.g., medications the resident takes), and the like. These attributes can then be processed as input to the acuity model, which generates an acuity score (which may be a single value or a collection of values) and/or classification (e.g., low, moderate, or high) indicating the acuity of the resident. As used herein, acuity refers to the intensity or amount of care that the resident or patient requires. Generally, higher acuity residents have higher needs, thereby requiring additional resources and/or staffing. In some embodiments, the acuity model can additionally be used to identify changes in acuity (e.g., over time), as well as identifying the specific attribute(s) that are causing a heightened acuity. In these ways, the acuity model can help drive specific and targeted interventions and assistance to improve resident outcomes and reduce wasted resources and computational expense.

### Example Workflow for Generating Acuity Models Using Historical Data

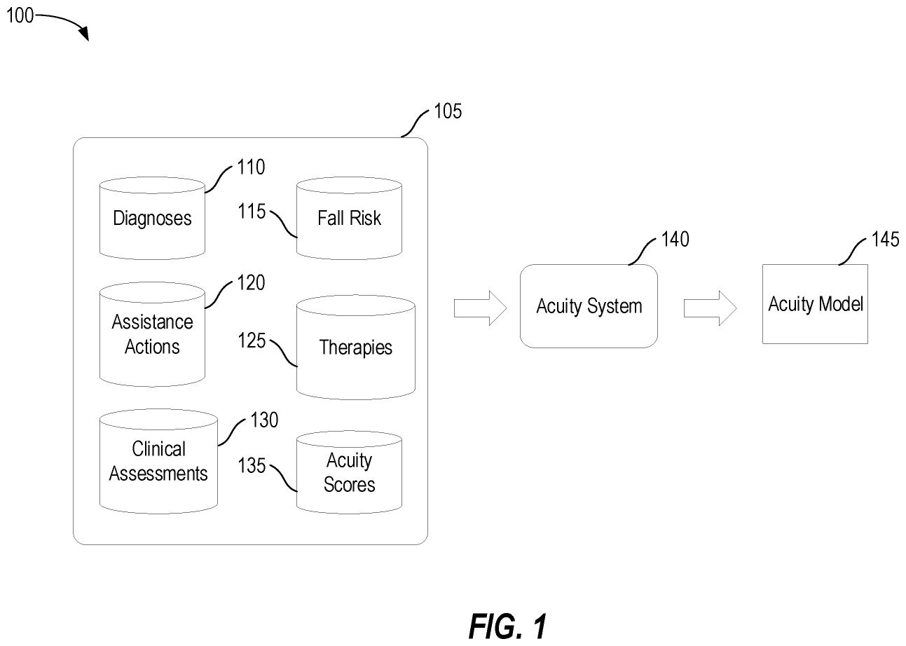

FIG. 1 depicts an example workflow 100 for generating acuity models based on historical data. In the illustrated workflow 100, a set of historical data 105 is evaluated by an acuity system 140 to generate one or more acuity models 145. In embodiments, the acuity system 140 may be implemented using hardware, software, or a combination of hardware and software. The historical data 105 generally includes data or information associated with one or more users (also referred to as patients or residents) from one or more prior points in time. That is, the historical data 105 may include, for one or more residents, a set of one or more snapshots of the resident's characteristics or attributes at one or more points in time. For example, the historical data 105 may include attributes for a set of residents residing in one or more long-term care facilities. In some embodiments, the historical data 105 includes indications of when any of the resident attributes changed (e.g., records indicating the updated attributes whenever a change occurs). The historical data 105 may generally be stored in any suitable location. For example, the historical data 105 may be stored within the acuity system 140, or may be stored in one or more remote repositories, such as in a cloud storage system.

In the illustrated example, the historical data 105 includes, for each resident reflected in the data, a set of one or more diagnoses 110, fall risk data 115, indications of assistance actions 120, therapies 125, clinical assessments 130, and acuity score(s) 135. In some embodiments, the acuity scores 135 are defined by a user (e.g., a caregiver, clinician, nurse, doctor, and the like) based on the resident's attributes at the time. That is, for a given resident, the user may review the resident attributes at a given time, and assign an acuity score 135 for that time. In this way, a single resident may have multiple acuity scores 135 (e.g., as the resident's other attributes change). In some embodiments, the data contained within the diagnoses 110, fall risk 115, assistance actions 120, therapies 125, and clinical assessments 130 are associated with timestamps or other indications of the relevant time or period for the data. In this way, the acuity system 140 can identify the corresponding acuity score 135 at the proper time (or, in some embodiments, can identify the relevant attributes for a given acuity score 135 associated with a given time).

In some embodiments, the historical data 105 may be collectively stored in a single data structure. For example, the diagnoses 110, fall risk 115, assistance actions 120, therapies 125, clinical assessments 130, and acuity scores 135 may each be represented in a resident profile (with indications of any changes over time), or as a sequence of structures (e.g., a set of profiles or forms, each corresponding to a particular point or window in time and containing attributes and an acuity score for that time). In some portions of the present discussion, the various components of the historical data 105 are described with reference to a single resident for conceptual clarity (e.g., diagnoses 110 of a single resident). However, it is to be understood that the historical data 105 can generally include such data for any number of residents.

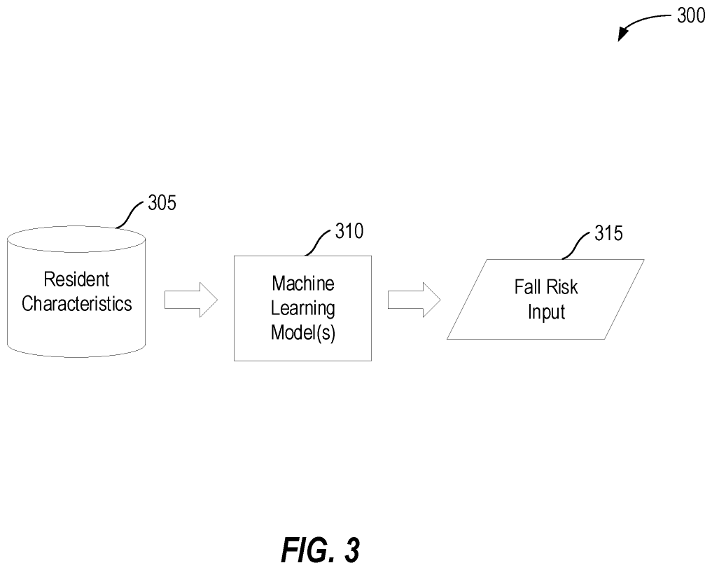

As discussed in more detail below with reference to FIG. 3, the fall risk 115 generally corresponds to a predicted likelihood that the resident will fall, and/or a predicted severity of the fall (if one occurs). In some embodiments, the fall risk 115 is generated using one or more machine learning models trained to generate the likelihood and/or severity of falls based on various patient characteristics and attributes.

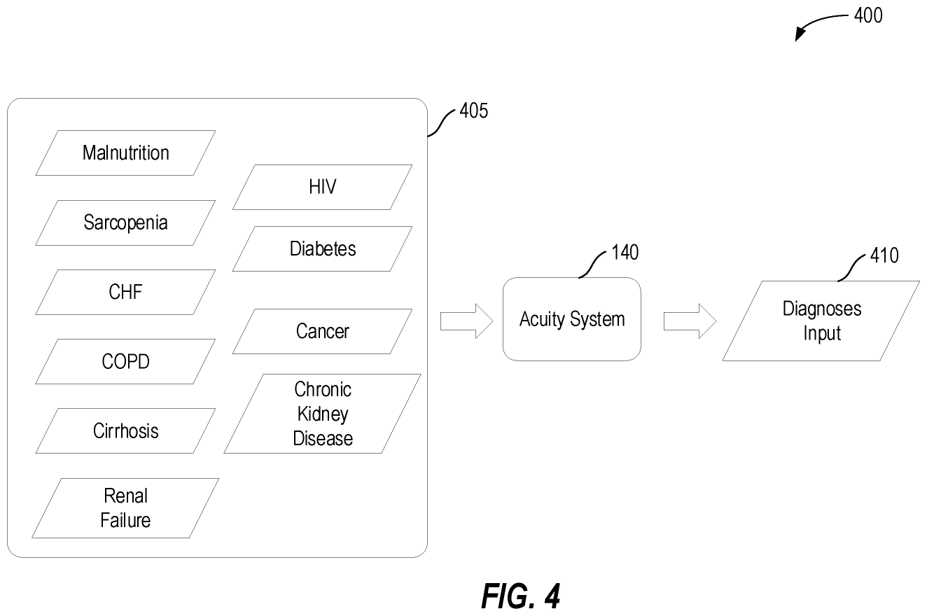

As discussed in more detail below with reference to FIG. 4, the diagnoses 110 generally correspond to a set of one or more specified diagnoses or disorders, and the data indicates, for each specified disorder, whether the corresponding resident has been diagnosed with the specified disorder. In some embodiments, the diagnoses 110 are curated or selected based on their impact on resident acuity. For example, in one aspect, a user (e.g., a clinician) may manually specify disorders that have a high impact on acuity. In some embodiments, some or all of the diagnoses may be inferred or learned (e.g., using one or more feature selection techniques). That is, one or more machine learning models or feature selection algorithms may be used to identify specific disorders that have a high impact on the acuity score 135 of the resident.

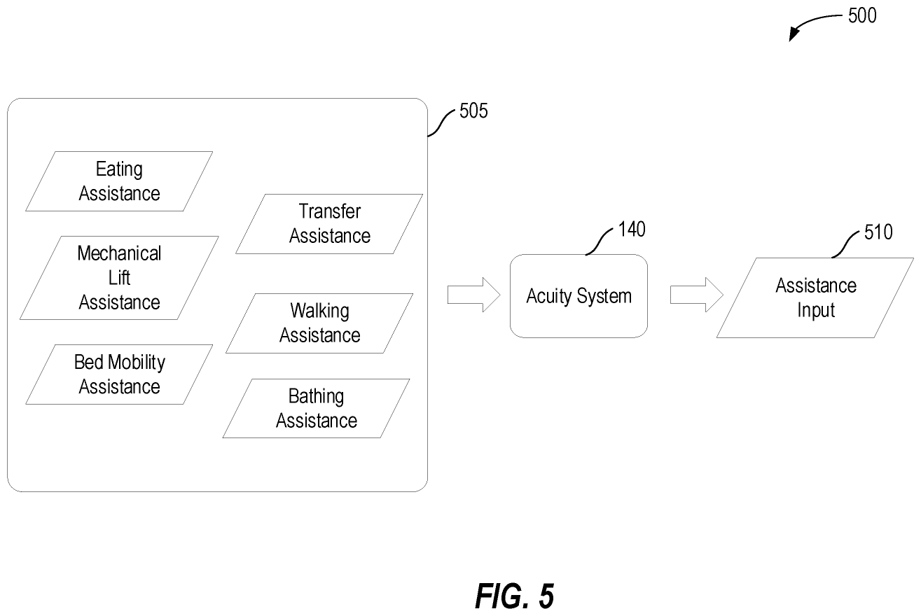

As discussed in more detail below with reference to FIG. 5, the assistance actions 120 generally correspond to a set of one or more specified point-of-care or assistance actions that can be performed by caregivers. The assistance actions 120 can generally indicate, for each specified action, whether the corresponding resident receives (or needs) the specified assistance. In some embodiments, the assistance actions 120 are curated or selected based on their impact on resident acuity. For example, in one aspect, a user (e.g., a clinician) may manually specify assistance actions that have a high impact on acuity (e.g., that require substantial caregiver time). In some embodiments, some or all of the assistance actions may be inferred or learned (e.g., using one or more feature selection techniques). That is, one or more machine learning models or feature selection algorithms may be used to identify specific actions that have a high impact on the acuity score 135 of the resident.

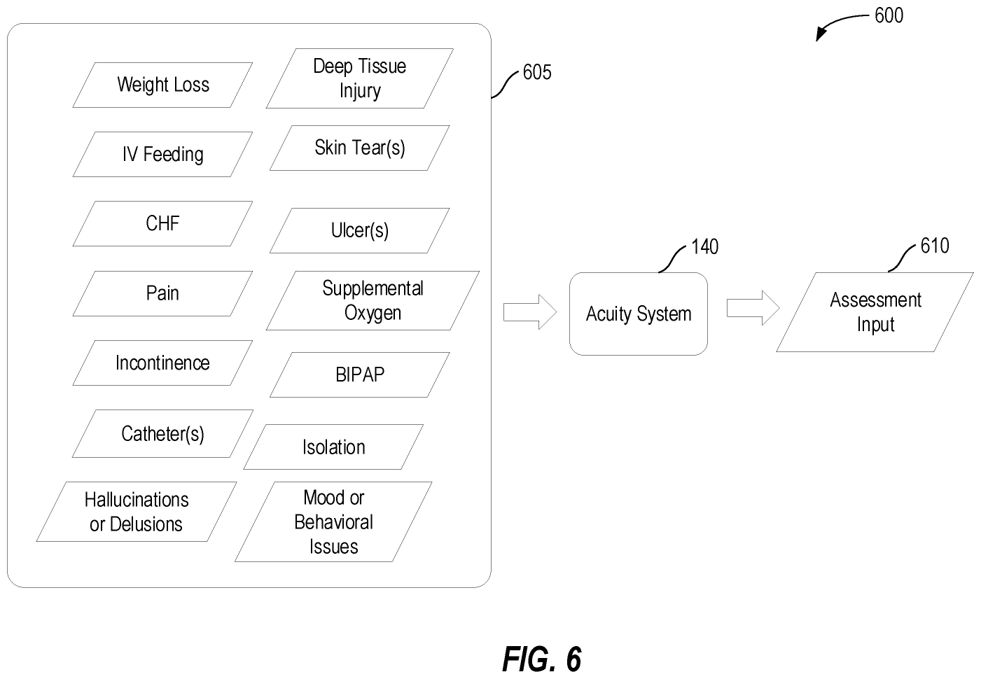

As discussed in more detail below with reference to FIG. 6, the clinical assessments 130 generally correspond to a set of one or more specified assessments (e.g., prepared by a caregiver or clinician) of conditions relating to the functional state of the resident. The clinical assessments 130 can generally indicate, for each specified condition, whether the corresponding resident has been assessed with the specified condition. For example, the clinical assessments 130 may indicate whether the resident has been assessed with skin tear(s), pain, and the like. In some embodiments, the clinical assessments 120 are curated or selected based on their impact on resident acuity. For example, in one aspect, a user (e.g., a clinician) may manually specify assessments or conditions that have a high impact on acuity. In some embodiments, some or all of the conditions or assessments may be inferred or learned (e.g., using one or more feature selection techniques). That is, one or more machine learning models or feature selection algorithms may be used to identify specific conditions or assessments that have a high impact on the acuity score 135 of the resident.

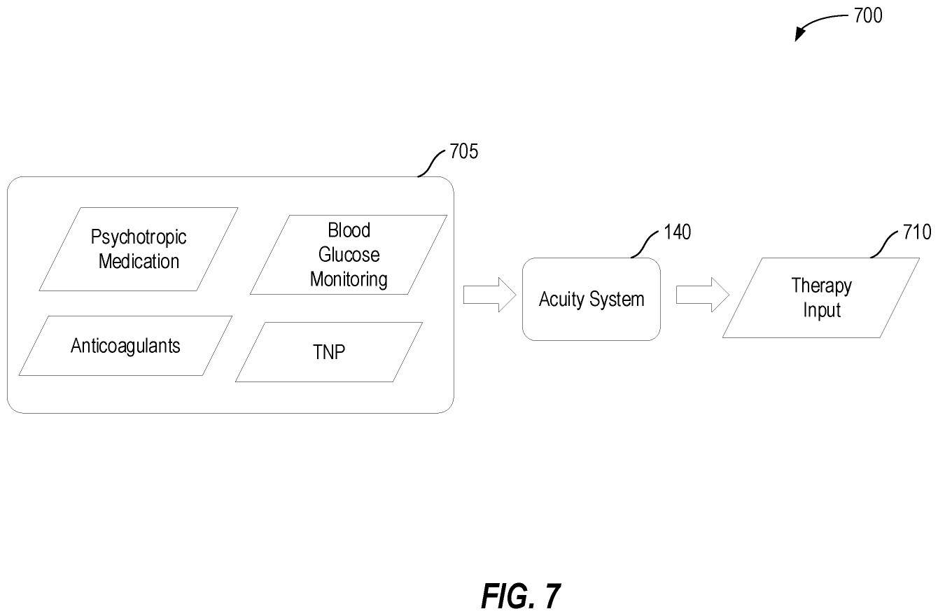

As discussed in more detail below with reference to FIG. 7, the therapies 125 generally correspond to a set of one or more specified therapies (e.g., medications or other interventions). The therapies 125 can generally indicate, for each specified medication, therapy, or intervention, whether the corresponding resident receives (or should receive) the medication, therapy, or intervention. In some embodiments, the therapies 125 are curated or selected based on their impact on resident acuity. For example, in one aspect, a user (e.g., a clinician) may manually specify interventions or therapies that have a high impact on acuity (or are reflective of high acuity needs). In some embodiments, some or all of the medications or therapies may be inferred or learned (e.g., using one or more feature selection techniques). That is, one or more machine learning models or feature selection algorithms may be used to identify specific therapies or medications that have a high impact on the acuity score 135 of the resident (or are otherwise correlated with high acuity).

Although the illustrated historical data 105 includes several specific components including diagnoses 110, fall risk 115, assistance actions 120, therapies 125, and clinical assessments 130, in some embodiments, the historical data 105 used by the acuity system 140 may include fewer components (e.g., a subset of the illustrated examples) or additional components not depicted. Additionally, though the illustrated example provides general groupings of attributes to aid understanding (e.g., grouping attributes as diagnoses, assistance actions, and the like), in some embodiments, the historical data 105 may be represented using any number of groups. That is, the individual attributes (e.g., individual diagnoses) may simply be used as input to the acuity system 140, without reference to any larger grouping or component.

Additionally, though the above discussion relates to receiving specified sets of data (e.g., specified diagnoses), in some aspects, the acuity system 140 may receive a broader set of data (e.g., all diagnoses of the residents) and select which subset of the data to consider (e.g., using feature selection techniques, as discussed above, or based on the defined set of features to be used).

As illustrated, the acuity model 140 generates an acuity model 145 based on the historical data 105. The acuity model 145 generally specifies a set of weights for the various features or attributes of the historical data 105. In some embodiments, the acuity model 145 specifies weights specifically for each individual feature (e.g., for each diagnosis in the set of specified diagnoses 110). For example, a diagnosis of diabetes may be associated with a lower weight than a diagnosis of renal failure. Similarly, in some embodiments, the acuity model 145 specifies different weights depending on the severity of the feature (e.g., depending on the severity of the renal failure). In some embodiments, the acuity model 145 specifies weights for groups of features (e.g., a first weight for the diagnoses 110, a second weight for the fall risk 115, and so on).

In at least one embodiment, the acuity model 145 can specify weights for one or more individual features, as well as weights for one or more broader categories. For example, the various diagnoses 110 may be weighted (e.g., with a first weight for sarcopenia and a second weight for cirrhosis) to generate an overall score or value for the diagnoses 110 input (e.g., a weighted sum or average). This value for the diagnoses 110 input can then be weighted (along with the other groups of input data) to generate an overall acuity for the resident (e.g., using a weighted sum or average).

In some embodiments, the specific features considered by the acuity model 145 (e.g., the specific diagnoses) and/or the weights used by the acuity model 145 (e.g., the weights for each specific diagnosis and/or input component) are manually defined and curated. For example, the specific features may be defined by a subject-matter expert, and the weights of each may similarly be defined manually or derived using statistical analysis. In other embodiments, the specific features and/or specific weights are learned during a training phase.

For example, the acuity system 140 may process the historical data 105 for a given resident at a given time as input to the acuity model 145, and compare the generated acuity to the ground-truth acuity score 135. The difference between the generated and actual acuity can be used to refine the weights of the acuity model 145, and the model can be iteratively refined (e.g., using data from multiple residents and/or multiple points in time) to generate an accurate acuity score.

In some embodiments, the acuity system 140 can generate multiple acuity models 145. For example, a separate acuity model 145 may be generated for each facility (e.g., with a unique model for each specific long-term residential care facility), or for each region (e.g., with a unique model for each country). In other embodiments, the acuity system 140 generates a universal acuity model 145.

In some embodiments, the acuity system 140 outputs the acuity model 145 to one or more other systems for use. That is, the acuity system 140 may distribute the acuity model 145 to one or more downstream systems, each responsible for one or more facilities. For example, the acuity system 140 may deploy the acuity model 145 to one or more servers associated with specific care facilities, and these servers may use the model to evaluate acuity for residents at the specific facility. In at least one embodiment, the acuity system 140 can itself use the acuity model to evaluate resident acuity across one or more locations.

### Example Workflow for Generating Acuity Scores and Interventions Using Acuity Models

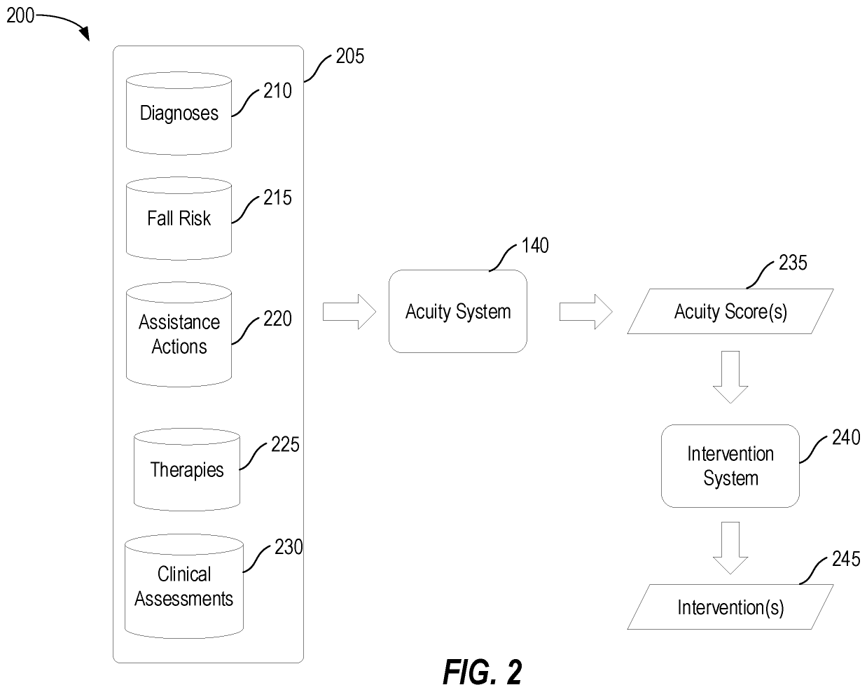

FIG. 2 depicts an example workflow 200 for generating acuity scores and interventions using acuity models.

In the illustrated workflow 200, a set of resident data 205 is evaluated by an acuity system 140 using one or more acuity models (e.g., acuity model 145 of FIG. 1) to generate one or more acuity score(s) 235. In embodiments, the acuity system 140 may be implemented using hardware, software, or a combination of hardware and software. In some embodiments, the acuity system 140 that uses the acuity model(s) is the same as the system that generates the acuity model. In other aspects, as discussed above, the acuity system 140 may differ from the system that generated the model.

The resident data 205 generally includes data or information associated with one or more users (also referred to as patients or residents). That is, the resident data 205 may include, for one or more residents, a set of one or more snapshots of the resident's characteristics or attributes at one or more points in time. For example, the resident data 205 may include attributes for a set of residents residing in one or more long-term care facilities. The resident data 205 may generally be stored in any suitable location. For example, the resident data 205 may be stored within the acuity system 140, or may be stored in one or more remote repositories, such as in a cloud storage system. In at least one embodiment, the resident data 205 is distributed across multiple data stores.

In the illustrated example, the resident data 205 includes, for each resident reflected in the data, a set of one or more diagnoses 210, fall risk data 215, indications of assistance actions 220, therapies 225, and clinical assessments 230. In some embodiments, the data contained within the diagnoses 210, fall risk 215, assistance actions 220, therapies 225, and clinical assessments 230 are associated with timestamps or other indications of the relevant time or period for the data. In this way, the acuity system 140 can generate a corresponding acuity score 235 based on the relevant attributes at a given time.

As discussed above and in more detail below with reference to FIG. 3, the fall risk 115 generally corresponds to a predicted likelihood that the resident will fall, and/or a predicted severity of the fall (if one occurs). As discussed above and in more detail below with reference to FIG. 4, the diagnoses 210 generally include information relating to a set of one or more specified diagnoses or disorders with respect to one or more residents. As discussed above and in more detail below with reference to FIG. 5, the assistance actions 220 generally include information relating to whether one or more specified point-of-care or assistance actions are needed by the resident(s). As discussed above and in more detail below with reference to FIG. 6, the clinical assessments 230 generally include information relating to whether one or more specified conditions (e.g., noted by a caregiver or clinician) have been noted for the resident(s). As discussed above and in more detail below with reference to FIG. 7, the therapies 225 generally include information relating to whether the resident(s) receive one or more specified medications, therapies, or other interventions.

Although the illustrated resident data 205 includes several discrete components for conceptual clarity, in some embodiments, the resident data 205 used by the acuity system 140 may include fewer components (e.g., a subset of the illustrated examples) or additional components not depicted. Additionally, though the illustrated example provides general groupings of attributes to aid understanding (e.g., grouping attributes as diagnoses, assistance actions, and the like), in some embodiments, the resident data 205 may be represented using any number of groups. That is, the individual attributes (e.g., individual diagnoses) may simply be used as input to the acuity system 140, without reference to any larger grouping or component.

Additionally, though the above discussion relates to receiving specified sets of data (e.g., specified diagnoses), in some aspects, the acuity system 140 may receive a broader set of data (e.g., all diagnoses of the residents) and select which subset of the data to consider (e.g., based on the features specified in the acuity model).

In the illustrated example, the resident data 205 is used to generate one or more acuity scores 235. The acuity scores 235 can generally include one or more scores for one or more residents. For example, in one aspect, the acuity scores 235 can include one score for each respective resident of a plurality of residents (e.g., in a single facility). In one aspect, the acuity scores 235 can include multiple scores for a single resident (or for reach resident), where each score is associated with a corresponding point or window in time (with a corresponding set of attributes from the resident data 205).

As discussed above, the acuity system 140 can generate the acuity scores 235 by identifying or extracting the relevant resident attributes from the resident data 205 (e.g., the relevant diagnoses, as indicated by the acuity model), and processing these attributes using the weights and architecture of the acuity model to generate an overall acuity score 235 for the resident based on the attributes. In some embodiments, the acuity score 235 can additionally or alternatively include an acuity classification or category (e.g., low, moderate, or high), determined based on one or more threshold values for the acuity score.

In some embodiments, the acuity system 140 can generate a new acuity score 235 for each resident periodically (e.g., daily). In some embodiments, the acuity system 140 generates a new acuity score 235 whenever new data becomes available (or when the resident data 205 changes). For example, when a new resident enters the facility, the acuity system 140 may use their current attributes to generate an initial acuity score. As another example, whenever a resident's attributes change (e.g., due to a newly-received diagnosis and/or a removed diagnosis, an updated fall risk, a newly-added or removed assistance action, and the like), the acuity system 140 may automatically detect the change and generate an updated acuity score 235.

In embodiments, the acuity scores 235 can be used for a wide variety of applications. In some embodiments, the acuity scores 235 are used to define or predict future resource allocations and care plans. For example, based on the acuity scores 235 (which may include aggregate scores for a group of residents), a user (or an automated system) may reallocate healthcare resources to best serve those with high acuity.

In the illustrated example, the acuity scores 235 are optionally provided to an intervention system 240, which can generate one or more interventions 245 based on the acuity scores 235. In some embodiments, the intervention system 240 can identify changes in the acuity score 235 of each resident. In one such embodiment, one or more of the generated interventions 245 may include alerts when the acuity score of a given resident changes (e.g., when it increases). For example, when new data is received for a given resident, and this new data causes the resident's acuity score 235 to increase, the intervention system 140 may transmit an alert (e.g., to one or more clinicians). In some embodiments, the alert may include information relating to the change, such as what caused the score to increase, when the increased occurred, the magnitude of the increase, and the like.

In at least one embodiment, the alert may include instructions or suggestions with respect to specific prophylactic interventions 245 for the resident, such as increased monitoring or check-ins by caregivers, modified meal content and/or schedule, renewed or more frequent clinical assessments for changing conditions, and the like. In some examples, the interventions 245 can include transporting or relocating the resident (e.g., to move them closer to a nursing station), to suggest or provide specialized care based on the underlying risk factors, to allocate (or reallocate) resources (such as staffing or medical resources) based on the acuity, and the like.

Advantageously, the automatically generated acuity scores 235 and/or interventions 245 can significantly improve the outcomes of the residents, helping to prevent further deterioration and significantly reducing harm, such as due to insufficient care or rehospitalization. Additionally, the autonomous nature of the acuity system 140 enables improved computational efficiency and accuracy, as the acuity scores 235 and/or interventions 245 are generated objectively (as opposed to the subjective judgment of clinicians or other users), as well as quickly and with minimal computational expense. That is, as the scores can be automatically updated whenever new data is available, users need not manually retrieve and review the relevant data (which incurs wasted computational expense, as well as wasted time for the user).

Further, in some embodiments, the acuity system 140 can regenerate acuity scores 235 during specified times (e.g., off-peak hours, such as overnight) to provide improved load balancing on the underlying computational systems. For example, rather than requiring caregivers to retrieve and review the resident data each morning to determine if anything significant occurred overnight or the previous day, the acuity system 140 can automatically identify such changes, and use the acuity model(s) to regenerate acuity scores 235 before the shift begins. This can transfer the computational burden, which may include both processing power of the storage repositories and access terminals, as well as bandwidth over one or more networks, to off-peak times, thereby reducing congestion on the system during ordinary (e.g., daytime) use and taking advantage of extra resources that are available during the non-peak (e.g., overnight) hours.

In these ways, embodiments of the present disclosure can significantly improve resident outcomes while simultaneously improving the operations of the computers and/or networks themselves (at least through improved and more accurate scores, as well as better load balancing of the computational burdens).

### Example Workflow for Generating Fall Risk Input for Acuity Evaluations

FIG. 3 depicts an example workflow 300 for using machine learning to generate fall risk input for acuity evaluations. In some embodiments, the workflow 300 is performed by an acuity system (such as the acuity system 140 of FIGS. 1 and 2). Generally, the workflow 300 is used to identify, extract, and/or generate a portion of the input data used by the acuity system. For example, the data may be historical data used to generate an acuity model, or may be current data that is processed by an acuity model to generate an acuity score.

In the illustrated example, a set of resident characteristics 305 are received for processing. The resident characteristics 305 can generally include various characteristics and attributes of any number of residents, such as their demographics, ages, mobility conditions, whether they use assistive devices (such as walkers or canes), and the like.

As illustrated, the resident characteristics 305 are processed using one or more trained machine learning models 310 to generate a corresponding fall risk input 315 for each resident. In some embodiments, the machine learning models 310 are trained by one or more other systems. Generally, the fall risk models are trained based on historical data to generate predictions relating to potential falls for the resident. For example, the generated fall risk input 315 may include a likelihood of the resident falling, a timeline of when a fall is expected, a predicted severity of the future fall, and the like.

In some embodiments, the machine learning models 310 include a hybrid model having one or more static portions (e.g., statistically-defined weights) and one or more dynamic or learned portions (e.g., a portion using trained weights derived during machine learning training). In some aspects, the machine learning models 310 may generally include pre-trained models trained and/or used by other systems. For example, in one embodiment, a separate system may train and use the machine learning models 310 to generate fall risk scores, which can then be received and used by the acuity system to generate fall risk input 315.

As discussed above, the fall risk input 315 can generally be used to generate acuity models (e.g., if it is historical data and the resident has an associated acuity score for the time), or as input to an acuity model to generate a new acuity score for the resident. For example, the fall risk input 315 may be included as the fall risk data 115 in FIG. 1, and/or the fall risk data 215 of FIG. 2.

By using such machine learning-generated fall risk input 315, the acuity system is able to generate more accurate acuity scores (enabling improved caregiver response) with reduced computational burden (e.g., because the scores can be objectively determined when the data becomes available, rather than frequently revisited or changed manually).

### Example Workflow for Generating Diagnoses Input for Acuity Evaluations

FIG. 4 depicts an example workflow 400 for generating diagnoses input for acuity evaluations. In some embodiments, the workflow 400 is performed by an acuity system (such as the acuity system 140 of FIGS. 1 and 2). Generally, the workflow 400 is used to identify, extract, and/or generate a portion of the input data used by the acuity system. For example, the data may be historical data used to generate an acuity model, or may be current data that is processed by an acuity model to generate an acuity score.

As illustrated, a set of resident data 405 are received for processing by the acuity system 140. In embodiments, the resident data 405 may include data for a single resident (e.g., to be used to generate an acuity score for the resident), or data for multiple residents (e.g., to be used to generate an acuity model). In the illustrated example, the resident data 405 corresponds to information relating to one or more diagnoses. Specifically, the resident data 405 indicates whether the resident (or each resident, if multiple residents are reflected in the resident data 405) has any of the specified diagnoses.

In some embodiments, as discussed above, the specified diagnoses are defined manually (e.g., by a clinician). These diagnoses may be selected for inclusion in the acuity model due to their impact on resident acuity. In other embodiments, the specified diagnoses may be inferred or derived using feature selection, machine learning, and the like. Additionally, though the illustrated example depicts receiving data specifically for the indicated diagnoses, in some aspects, the resident data 405 can include information relating to any number of diagnoses, and the acuity system 140 can select or extract the data for the relevant diagnoses (e.g., as indicated in the acuity model).

In the illustrated example, the specific diagnoses 405 include a diagnosis of malnutrition (e.g., indicated by International Classification of Diseases (ICD)-10 codes from E40 through E46, and/or R62.7), a diagnosis of sarcopenia (e.g., indicated by ICD-10 code M62.84), a diagnosis of congestive heart failure (CHF) (e.g., indicated by ICD-10 code ISO), a diagnosis of chronic obstructive pulmonary disease (COPD) (e.g. indicated by ICD-10 code J44), a diagnosis of cirrhosis (e.g., indicated by ICD-10 code K70.30, K70.31, and/or any K74 code), a diagnosis of renal failure or end-stage renal disease (e.g., indicated by ICD-10 code N18.6, I12.0, I13.11, and/or I13.2), a diagnosis of human immunodeficiency virus (HIV) (e.g., indicated by ICD-10 code B20 and/or Z21), a diagnosis of diabetes and/or diabetes mellitus—type 2 (uncontrolled) (e.g., indicated by ICD-10 code E08.10, E08.11, and/or any E11 code), a diagnosis of cancer (e.g., indicated by minimum dataset (MDS) code 10100 or 00100.2 (A or B)), and a diagnosis of chronic kidney disease (e.g., indicated by ICD-10 code I12.9, E13.22, E09.22, E11.22, E08.22, and/or any N18 code).

The illustrated diagnoses are provided as conceptual examples of diagnoses that affect acuity, and are not intended to limit the scope of the present disclosure in any way. In the illustrated example, the acuity system 140 can evaluate the resident data 405 to determine, for each specified diagnosis or disorder, whether the resident has been diagnosed with the disorder. For example, the resident data 405 may include electronic health records (EHR) data for the resident, and the acuity system 140 may search it (e.g., using the above-discussed ICD-10 and MDS codes) to determine whether the resident has each disorder. In some embodiments, this includes determining whether the resident was ever diagnosed with each disorder, whether the resident was recently diagnosed with them (e.g., within a defined time), whether the diagnosis is pertinent/the resident actively has the diagnosis (e.g., confirming whether an old diagnosis is still applicable), and the like.

Based on this analysis, the acuity system 140 can generate the diagnoses input 410 for the resident. As discussed above, this diagnoses input 410 may be used to generate an acuity model (e.g., corresponding to the diagnoses 110 of FIG. 1), and/or to generate an acuity score (e.g., corresponding to the diagnoses 210 of FIG. 2).

### Example Workflow for Generating Assistance Input for Acuity Evaluations

FIG. 5 depicts an example workflow 500 for generating assistance input for acuity evaluations. In some embodiments, the workflow 500 is performed by an acuity system (such as the acuity system 140 of FIGS. 1 and 2). Generally, the workflow 500 is used to identify, extract, and/or generate a portion of the input data used by the acuity system. For example, the data may be historical data used to generate an acuity model, or may be current data that is processed by an acuity model to generate an acuity score.

As illustrated, a set of resident data 505 are received for processing by the acuity system 140. In embodiments, the resident data 505 may include data for a single resident (e.g., to be used to generate an acuity score for the resident), or data for multiple residents (e.g., to be used to generate an acuity model). In the illustrated example, the resident data 505 corresponds to information relating to one or more assistance events or actions (also referred to in some aspects as point-of-care actions or events). Specifically, the resident data 505 indicates whether the resident (or each resident, if multiple residents are reflected in the resident data 505) requires or receives any of the specified assistances.

In some embodiments, as discussed above, the specified assistance events are defined manually (e.g., by a clinician). These actions may be selected for inclusion in the acuity model due to their impact on resident acuity. In other embodiments, the specified actions may be inferred or derived using feature selection, machine learning, and the like. Additionally, though the illustrated example depicts receiving data specifically for the indicated actions, in some aspects, the resident data 505 can include information relating to any number of actions and needs, and the acuity system 140 can select or extract the data for the relevant assistance actions (e.g., as indicated in the acuity model).

In the illustrated example, the specific assistance events can include a need for one or more assistance and daily living (ADL) actions, such as eating assistance (e.g., help with eating), bed mobility assistance (e.g., help getting into or out of bed), transfer assistance (e.g., help with transferring from seated to standing positions, and vice versa), walking assistance (e.g., help with walking in the room, in corridors, and the like), bathing assistance (e.g., help with bathing), and the like. In the illustrated example, the specific actions can also include a need for mechanical lift assistance (e.g., if a mechanical lift is needed to aid the resident in getting into or out of bed, getting into or out of seated positions, and the like), as this may further increase acuity. In some aspects, these ADL needs can be indicated by point-of-care notes from clinicians or caregivers.

The illustrated assistance actions are provided as conceptual examples of needed actions that affect acuity, and are not intended to limit the scope of the present disclosure in any way. In the illustrated example, the acuity system 140 can evaluate the resident data 505 to determine, for each specified assistance event, whether caregivers provide the indicated assistance to the resident (or whether the resident generally needs the assistance). For example, the resident data 505 may include electronic health records (EHR) data or clinical notes for the resident, and the acuity system 140 may search this data to determine whether the resident receives the indicated assistance. In an embodiment, this can include determining whether the resident currently receives the assistance, and/or has recently requested the assistance (even if it is not-yet provided).

Based on this analysis, the acuity system 140 can generate the assistance input 510 for the resident. As discussed above, this assistance input 510 may be used to generate an acuity model (e.g., corresponding to the assistance actions 120 of FIG. 1), and/or to generate an acuity score (e.g., corresponding to the assistance actions 220 of FIG. 2).

### Example Workflow for Generating Clinical Assessment Input for Acuity Evaluations

FIG. 6 depicts an example workflow 600 for generating clinical assessment input for acuity evaluations. In some embodiments, the workflow 600 is performed by an acuity system (such as the acuity system 140 of FIGS. 1 and 2). Generally, the workflow 600 is used to identify, extract, and/or generate a portion of the input data used by the acuity system. For example, the data may be historical data used to generate an acuity model, or may be current data that is processed by an acuity model to generate an acuity score.

As illustrated, a set of resident data 605 are received for processing by the acuity system 140. In embodiments, the resident data 605 may include data for a single resident (e.g., to be used to generate an acuity score for the resident), or data for multiple residents (e.g., to be used to generate an acuity model). In the illustrated example, the resident data 605 corresponds to information relating to one or more conditions. Specifically, the resident data 605 indicates whether the resident (or each resident, if multiple residents are reflected in the resident data 605) have been assessed as having any of the specified conditions.

In some embodiments, as discussed above, the specified conditions are defined manually (e.g., by a clinician). These conditions may be selected for inclusion in the acuity model due to their impact on resident acuity. In other embodiments, the specified conditions may be inferred or derived using feature selection, machine learning, and the like. Additionally, though the illustrated example depicts receiving data specifically for the indicated conditions, in some aspects, the resident data 605 can include information relating to any number of conditions and assessments, and the acuity system 140 can select or extract the data for the relevant conditions (e.g., as indicated in the acuity model).

In the illustrated example, the specific conditions include an assessment of weight loss (e.g., indicated by MDS code K0300), an assessment that parenteral or intravenous (IV) feeding is used or needed (e.g., indicated by MDS code K0510.2 (A or B)), an assessment or diagnosis of CHF (e.g., indicated by ICD-10 code I50), an assessment indicating that the resident experiences some type of pain (e.g., indicated by MDS codes O0100.2.J, J0850, and/or J0400), an assessment of incontinence (e.g., indicated by MDS codes H0300, and/or H0400), an assessment indicating that the resident has a catheter and/or ostomy (e.g., indicated by MDS code H0100 (A, B, C, or D)), an assessment that the resident suffers from hallucinations and/or delusions (e.g., indicated by MDS code E0100 (A or B)), an assessment of one or more deep tissue injuries (e.g., indicated by MDS code M0300), an assessment of one or more skin tears or abrasions (e.g., indicated by MDS code M1040), an assessment of one or more ulcers (e.g., indicated by MDS codes M0300A1, M0300B1, M0300C1, and/or M0300D1), an assessment of a need for supplemental oxygen (e.g., indicated by MDS code O01002C), an assessment or indication that the resident uses or needs bilevel positive airway pressure (BIPAP) support (e.g., indicated by MDS code O01002G), an assessment indicating that the resident is in (or needs to be in) isolation, such as due to infection (e.g., indicated by MDS code O0100.M.2), and/or an assessment of mood and/or behavioral issues (e.g., indicated by MDS codes D0200, D0500, E0200, and/or E0900).

The illustrated assessments and conditions are provided as conceptual examples of conditions that affect acuity, and are not intended to limit the scope of the present disclosure in any way. In the illustrated example, the acuity system 140 can evaluate the resident data 605 to determine, for each specified condition or assessment, whether the resident has been assessed with the condition. For example, the resident data 605 may include electronic health records (EHR) data and/or MDS data for the resident, and the acuity system 140 may search it (e.g., using the above-discussed MDS codes) to determine whether the resident has been assessed with or for each condition. In some embodiments, this includes determining whether the resident currently has the condition (e.g., reflected in the most-recently available data for the resident).

Based on this analysis, the acuity system 140 can generate the assessment input 610 for the resident. As discussed above, this assessment input 610 may be used to generate an acuity model (e.g., corresponding to the clinical assessments 130 of FIG. 1), and/or to generate an acuity score (e.g., corresponding to the clinical assessments 230 of FIG. 2).

### Example Workflow for Generating Therapy Input for Acuity Evaluations

FIG. 7 depicts an example workflow 700 for generating therapy input for acuity evaluations. In some embodiments, the workflow 700 is performed by an acuity system (such as the acuity system 140 of FIGS. 1 and 2). Generally, the workflow 700 is used to identify, extract, and/or generate a portion of the input data used by the acuity system. For example, the data may be historical data used to generate an acuity model, or may be current data that is processed by an acuity model to generate an acuity score.

As illustrated, a set of resident data 705 are received for processing by the acuity system 140. In embodiments, the resident data 705 may include data for a single resident (e.g., to be used to generate an acuity score for the resident), or data for multiple residents (e.g., to be used to generate an acuity model). In the illustrated example, the resident data 705 corresponds to information relating to one or more therapies (e.g., medications or other treatments) received by the resident(s). Specifically, the resident data 705 indicates whether the resident (or each resident, if multiple residents are reflected in the resident data 705) receive the specified therapies.

In some embodiments, as discussed above, the specified therapies are defined manually (e.g., by a clinician). These therapies may be selected for inclusion in the acuity model due to their impact on resident acuity. In other embodiments, the specified therapies may be inferred or derived using feature selection, machine learning, and the like. Additionally, though the illustrated example depicts receiving data specifically for the indicated therapies, in some aspects, the resident data 705 can include information relating to any number of therapies and medications, and the acuity system 140 can select or extract the data for the relevant therapies (e.g., as indicated in the acuity model).

In some embodiments, each of these therapies can be indicated in one or more orders or prescriptions associated with the resident. In the illustrated example, the specific therapies include an indication as to whether the resident needs or receives psychotropic medication (e.g., identified by searching for medications within one or more defined drug classes such as central nervous system agents, anorexigenic medications, anxiolytics, general anesthetics, psychotherapeutic agents, and the like), an indication as to whether the resident needs or receives anticoagulant medication (e.g., identified by drug class, such as antithrombotic agents), an indication as to whether the resident needs or receives blood glucose monitoring (e.g., indicated by orders for antidiabetic or antihypoglycemic medications), and/or an indication as to whether the resident needs or receives total (or partial) parenteral nutrition (TNP) (e.g., indicated by an order for national drug code (NDC) codes 00409577901, 00409323601, and/or 00409577911).

The illustrated therapies are provided as conceptual examples of therapies that affect acuity, and are not intended to limit the scope of the present disclosure in any way. In the illustrated example, the acuity system 140 can evaluate the resident data 705 to determine, for each specified therapy, whether the resident receives or needs the therapy. For example, the resident data 605 may include electronic health records (EHR) data and/or prescription orders for the resident, and the acuity system 140 may search the data to determine whether the resident has been prescribed the therapy. In some embodiments, this includes determining whether the resident currently uses the therapy (e.g., reflected in the most-recently available data for the resident).

Based on this analysis, the acuity system 140 can generate the therapy input 710 for the resident. As discussed above, this therapy input 710 may be used to generate an acuity model (e.g., corresponding to the therapies 125 of FIG. 1), and/or to generate an acuity score (e.g., corresponding to the therapies 225 of FIG. 2).

### Example Graphical User Interface for Acuity Evaluations

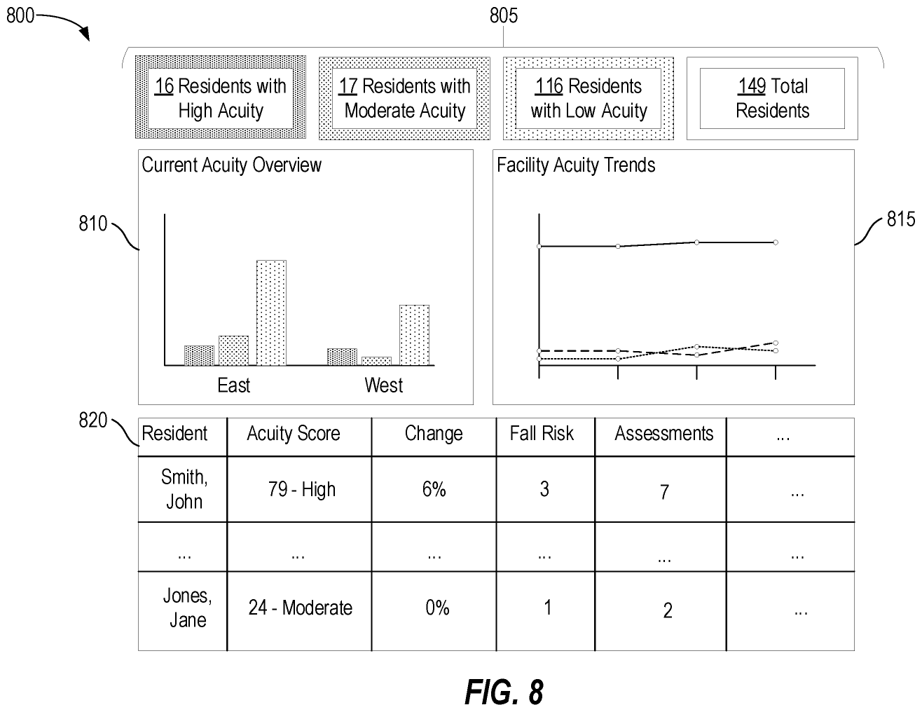

FIG. 8 depicts an example graphical user interface (GUI) 800 for acuity evaluations. In some embodiments, the GUI 800 may be generated and/or output by an acuity system (such as the acuity system 140 of FIGS. 1 and 2). The illustrated GUI 800 may be used to efficiently provide accurate acuity information for any number of residents in a facility. The GUI 800 is depicted as one illustrative example of a GUI, and the particular arrangement and content of the GUI may vary depending on the particular implementation.

In some embodiments, users (e.g., clinicians or caregivers) may use the GUI 800 to quickly review the acuity information for any residents under their care (or all residents in the facility), enabling more efficient transfer of the information and improved caregiving. For example, at the beginning of each shift, a caregiver may review the GUI 800 to quickly identify any residents with changed conditions (e.g., increased acuity), residents with high acuity, and the like.

In the illustrated example, a first portion 805 of the GUI 800 indicates the overall distribution of acuities within a facility. Specifically, in the illustrated example, the facility hosts 16 residents with high acuity (e.g., with generated acuity scores that are greater than or equal to a first threshold), 17 residents with a moderate acuity (e.g., with generated acuity scores that are less than the first threshold, but greater than or equal to a second threshold), and 116 residents with low acuity (e.g., with generated acuity scores that are less than the second threshold). Additionally, there are 149 total residents in the facility.

In some embodiments, the first portion 805 includes color coding, patterns, highlighting, or other visual indications of the acuities. For example, the residents with high acuity may be indicated using one color (e.g., red), while residents with moderate acuity are indicated using another, (e.g., orange or yellow) and residents with low acuity are indicated with yet another color (e.g., green or white). Generally, the first portion 805 can be used to derive a quick overview of the facility, which may inform resource allocations. For example, the user may note that a significant fraction of the total residents have high acuity, and thereby determine to allocate additional resources to the facility.

In the illustrated example, the GUI 800 also includes a portion 810 to provide an overview of the current acuity distributions in the facility. Specifically, the portion 810 indicates the number of residents having specific acuity levels in each section of the facility (e.g., in each building). In at least one embodiment, the portion 810 can be color-coded or can similarly include visual patterns to indicate the acuity distribution, as with the portion 805. For example, the color used to indicate the high acuity residents in the portion 805 can also be used in the corresponding section(s) of the portion 810.

In some embodiments, the portion 810 can be used to derive a quick overview of the distribution of residents in the facility, which may inform resource allocations. For example, the user may note that one building or section of the facility has a higher fraction of the high-acuity residents, and may thereby determine to allocate additional resources to that building or section.

In the illustrated example, the GUI 800, also includes a portion 815 to indicate acuity trends in the facility. Specifically, the portion 815 may indicate (e.g., using a line graph) how the total number of residents within each acuity band has changed over time. In at least one embodiment, the portion 815 can be color-coded or can similarly include visual patterns to indicate the acuity distribution, as with the portions 805 and 810. For example, the color used to indicate the high acuity residents in the portion 805 and 810 can also be used to indicate the corresponding line(s) of the portion 815.

In some embodiments, the portion 815 can be used to derive a quick overview of how acuity is changing in the facility, which may inform resource allocations and interventions. For example, the user may note that the number of high-acuity residents has been increasing, and may thereby determine to allocate additional resources, or to investigate what may be causing the increased acuity.

In the illustrated example, the GUI 800, also includes a portion 820 to indicate specific acuity scores and data for each specific resident. Specifically, the portion 820 may indicate, for each resident, the current acuity score and/or acuity level, recent changes in the acuity score or level, specific sub-scores for any of the components of the acuity score (e.g., a score for fall risk, a score for clinical assessments, and so on), and the like. In at least one embodiment, the portion 820 can be color-coded or can similarly include visual patterns to indicate the acuity distribution, as with the portions 805, 810, and 815. For example, the color used to indicate the high acuity residents in the portion 805, 810, and 815 can also be used to indicate the high acuity residents in the portion 820.

In some embodiments, the portion 820 can be used to quickly review the current state of any residents in the facility. In at least one embodiment, the portion 820 may enable filtering and/or sorting, such that the user can sort by high acuity, sort to show residents with changes in acuity at the top, and the like. Additionally, by reviewing the portion 820, the user can quickly and efficiently identify not only which residents have recently seen increased acuity, but also why these acuity scores have changed.

In at least one embodiment, the user may select any resident indicated in the portion 820 in order to review a resident-specific overview. For example, this overview may depict the resident's acuity over time (e.g., using a line graph) for the overall acuity scores, individual components of the acuity scores, and the like. This can allow the user to quickly identify what changes have occurred, and therefore select the best interventions to improve the resident outcomes.

### Example Method for Generating Acuity Models

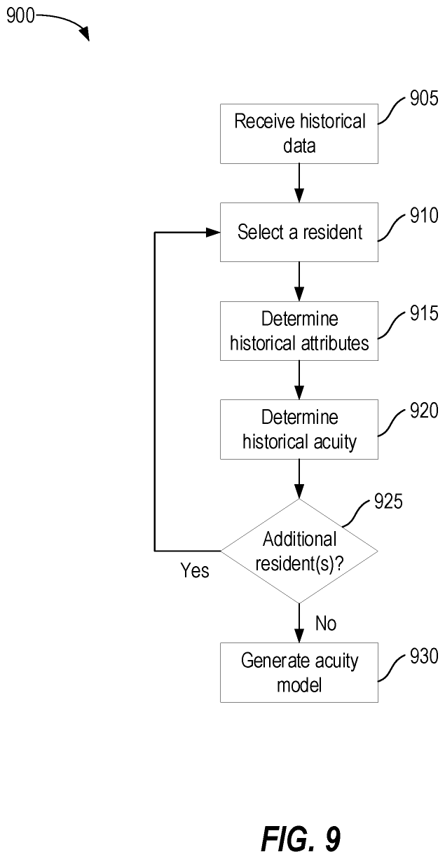

FIG. 9 is a flow diagram depicting an example method 900 for generating acuity models. In some embodiments, the method 900 is performed by an acuity system, such as the acuity system 140 of FIGS. 1 and 2.

At block 905, the acuity system receives historical data for one or more residents or patients. For example, as discussed above with reference to FIG. 1, the historical data may include may correspond to the historical data 105. In an embodiment, the received historical data can generally include data or information associated with one or more residents (also referred to as patients or users) from one or more prior points in time. That is, the historical data may include, for one or more residents, a set of resident characteristics or attributes at one or more points in time. In some embodiments, the historical data includes attributes for a set of residents residing in one or more long-term care facilities.

In some embodiments, the received historical data includes patient data corresponding to a defined set of features to be used to generate an acuity model. These features may include, for example, specified diagnoses, fall risk data, specified assistance actions or needs, specified clinical assessments and/or conditions, specified therapies (such as medications), and the like. In some embodiments, the historical data can include additional data beyond these features (e.g., information about all medications that one or more residents have been prescribed, regardless of the specific medications used in the acuity model). In one such embodiment, the acuity system can identify and extract the relevant attributes or data, based on the indicated features for the acuity model. In other embodiments, the received historical data may include only the specific data corresponding to the indicated features (e.g., another system may filter the historical data based on the features, thereby protecting data that is not needed to build the model).

In some embodiments, the historical data can also include one or more acuity scores for each resident reflected in the data. For example, for each set of attributes (e.g., for a given resident at a given time), the historical data may indicate a corresponding acuity score or category (e.g., assigned by a clinician). As discussed above, these acuity scores can generally indicate the amount or intensity of care needed for the resident. For example, some disorders require additional care, resulting in a higher acuity.

At block 910, the acuity system selects a resident reflected in the received historical data. That is, the acuity system can select a resident (e.g., randomly or pseudo-randomly) that has not-yet been evaluated. In some aspects, the acuity system selects a resident that has at least one set of attributes that has not-yet been evaluated. For example, the acuity system may select a resident that has at least one period or window of time for which the historical data indicates a set of attributes, but which the acuity system hasn't evaluated to generate the acuity model.

At block 915, the acuity system determines a set of historical attributes for a resident reflected in the historical data. For example, the acuity system may extract a set of attributes for a given point or window of time (e.g., randomly or pseudo-randomly) that has not-yet been evaluated. For example, the acuity system may identify the resident's current or pertinent diagnoses at the time, medications or other therapies the resident was receiving at the time, fall risk at the time, and the like.

At block 920, the acuity system then determines a historical acuity corresponding to the selected historical attributes. That is, the acuity system can determine what acuity was assigned to the resident based on the historical attributes. In some embodiments, this can include an acuity score (e.g., between zero and one hundred) indicating the resident's acuity, where higher scores indicate higher acuity (and therefore, higher needs). In some embodiments, the acuity system additionally or alternatively determines an acuity category (such as high acuity, medium acuity, or low acuity) for the resident. In at least one embodiment, the determined acuity is manually defined by a clinician (e.g., a caregiver that cared for the resident at the time corresponding to the historical attributes). In at least one embodiment, the historical acuity is determined or inferred based on other data, such as nursing time spent with the resident. This set of attributes and corresponding acuity can be used to build the acuity model, as discussed in more detail below.

At block 925, the acuity system determines whether there is at least one additional resident (or at least one resident with at least some data that has not-yet been evaluated) reflected in the historical data. If so, the method 900 returns to block 910. If all of the data has been evaluated (e.g., to identify pairs of data including historical attributes and a corresponding acuity), the method 900 continues to block 930.

At block 930, the acuity system generates an acuity model based on the above-identified pairs of data. That is, based on the respective acuity that was assigned to each respective set of historical attributes, the acuity system can generate a model specifying weights for each feature, such that the weights can be used to process newly-received attributes to generate an acuity score or category. In some embodiments, when generating the acuity model, the acuity system considers only the specified set of features (e.g., the specific diagnoses). In other embodiments, the acuity system may evaluate all available features. In one such embodiment, the eventual acuity model may exclude some features, and/or some features may be associated with low (or zero) weight (e.g., because they were found to be of little or no value in predicting acuity).

In some embodiments, the acuity model is generated using statistical analysis, resulting in a static model. For example, the acuity system may use statistical analysis to determine the impact or contribution of each feature, with respect to the final resident acuity. In some embodiments, the weights of each feature (and, in some cases, whether the feature is used at all) are defined based on this statistical analysis (e.g., based on the contribution of each feature). In this way, the acuity model can be used to generate new acuity scores and/or classifications based on newly-received attribute data.

In some embodiments, the acuity model is generated using machine learning. For example, the acuity system may use the historical attributes and acuity data as training exemplars (e.g., where each set of resident attributes is the input data, and the corresponding acuity is the target output). Using these exemplars, the acuity system can iteratively refine a machine learning model (e.g., using backpropagation in the case of a neural network). For example, the attributes can be provided as input to the model (e.g., with each attribute or feature having a corresponding input neuron) to generate an output acuity (score or category). This output can then be compared against the ground-truth label (determined at block 920), and the weights of the model can be refined based on this comparison (e.g., based on a loss computed using the generated acuity and actual acuity).

Regardless of the specific methods used to generate the acuity model, the acuity system can then deploy the model for use (locally or on one or more remote systems) to evaluate patient data and return corresponding acuity scores or measures.

### Example Method for Generating Acuity Scores Using Acuity Models

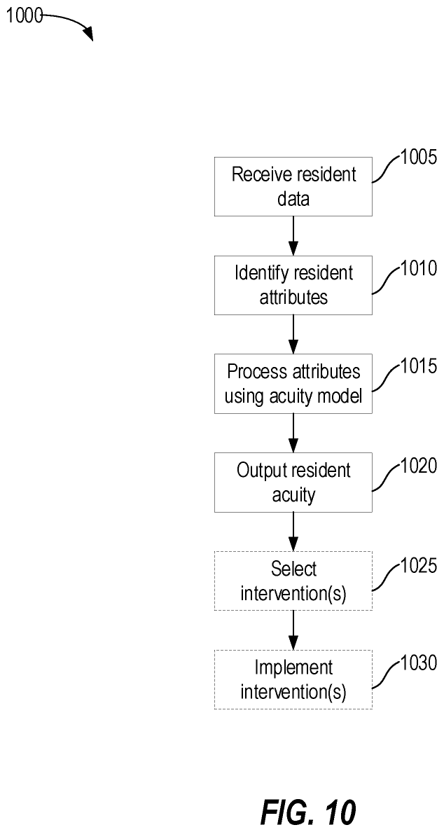

FIG. 10 is a flow diagram depicting an example method 1000 for generating acuity scores using acuity models. In some embodiments, the method 1000 is performed by an acuity system, such as the acuity system 140 of FIGS. 1 and 2.

At block 1005, the acuity system receives resident data. For example, as discussed above with reference to FIG. 2, the resident data may include may correspond to the resident data 205. In an embodiment, the received resident data can generally include data or information associated with a resident (also referred to as a patient or a user in some aspects). In some embodiments, the resident data includes attributes for a resident residing in a long-term care facility.

In an embodiment, the resident data can generally include information relating to attributes of the resident, such as a set of one or more diagnoses of the resident, fall risk data for the resident, indications of assistance actions needed by the resident, therapies or medications used by the resident, and clinical assessments of condition(s) of the resident. In some embodiments, the received resident data corresponds to current information for the resident. That is, the resident data may be the most-recent data for each feature.

In at least one aspect, the resident data is received because a change has occurred. That is, the resident data may be provided to the acuity system (e.g., using a push technique) based on determining that one or more of the attributes have changed since the last time the data was provided. In other embodiments, the acuity system can retrieve or request the resident data, and evaluate it to determine whether any attributes have changed. In at least one embodiment, if no attributes have changed (with respect to the relevant features used by the model), the acuity system can refrain from further processing of the data (e.g., refrain from generating a new acuity score), thereby reducing computational expense.

Similarly, if the data is only provided upon detecting a change, the acuity system need not review it at all, which also reduces computational expense of the system. Additionally, in some embodiments, the acuity system can receive only the updated data (as opposed to receiving or retrieving the entirety of the resident's data). That is, the storage systems may automatically transmit data when it is updated (or the acuity system may request any new or change data), enabling the acuity score to be revised based on the new data without the need to re-transmit the older data. This, again, reduces the computational expense (including bandwidth, if the data is stored remotely from the acuity system) of generating the scores.

In some embodiments, the received resident data includes patient data corresponding to a defined set of features to be used to generate an acuity model. These features may include, for example, specified diagnoses, fall risk data, specified assistance actions or needs, specified clinical assessments and/or conditions, specified therapies (such as medications), and the like. In some embodiments, the resident data can include additional data beyond these features (e.g., information about all medications that the resident has been prescribed, regardless of the specific medications used in the acuity model). In one such embodiment, the acuity system can identify and extract the relevant attributes or data, based on the indicated features for the acuity model. In other embodiments, the received resident data may include only the specific data corresponding to the indicated features (e.g., another system may filter the resident data based on the features, thereby protecting data that is not needed to build the model).

At block 1010, the acuity system identifies the set of relevant resident attributes, from the resident data, based on the specified features that are used by the acuity model. That is, the acuity system can extract, from the resident data, the relevant information for each feature. For example, if a specific diagnosis is included in the features, the acuity system may search the resident data using one or more diagnosis codes (e.g., ICD-10 codes) corresponding to the specific diagnosis. If the resident currently has the diagnosis, it can be indicated in the corresponding resident attribute (e.g., with a value indicating the presence of the diagnosis). If the diagnosis is not found, this attribute may be set to a defined value, such as a null value or a value of zero, to indicate that the resident does not have the feature.

At block 1015, the acuity system processes the identified/extracted attributes using the acuity model. As discussed above, the acuity model may specify a set of weights for input features. In some embodiments, as discussed above, the model may specify weights for individual features, for groups of features, or for both individual features and groups of features. For example, a “diagnosis” category may be assigned a first weight. To generate a score for the diagnosis category, the individual diagnoses specified within may each be associated with respective weights. That is, a diagnosis of one disorder may have a higher weight than a second disorder.

In this way, the acuity system can use the weights to generate a score for each group of features (if groups are used), such as by multiplying the weight of each feature with an indication as to whether the resident has the feature (or, in some aspects, a value indicating the severity of the feature). For example, in some embodiments, the system can use binary indications, where the resident either has the feature (e.g., a value of one) or does not (e.g., a value of zero). In some embodiments, the system can additionally or alternatively use non-binary values to indicate the severity (e.g., where a value of zero may indicate that the resident does not have the feature, a value of one indicates that the resident has a mild case, a value of two indicates a moderate case, and so on).

The resulting values for each weighted attribute can then be combined (e.g., summed) to generate an overall score with respect to the feature group. In an embodiment, the acuity system can similarly generate scores for each other group, and then aggregate these (potentially using a weighted sum, as discussed above) to generate an overall acuity score for the resident.

At block 1020, the acuity system outputs the resident acuity. In embodiments, this may include, for example, outputting it via a display or terminal (e.g., for a caregiver to review), such as via the GUI 800 of FIG. 8. In some embodiments, block 1020 includes outputting the acuity score to one or more other systems (e.g., a storage repository, or other systems that evaluate the acuity to inform allocation decisions), and the like.

At block 1025, the acuity system (or another system, such as the intervention system 240 of FIG. 2) can optionally select one or more interventions based on the generated acuity. That is, as discussed above, the system may select one or more prophylactic interventions to reduce potential harm, based on the current resident acuity. For example, based on an increase in acuity, the system may allocate additional resources to the resident, instruct a user (e.g., a caregiver) to care for the resident, and the like. In some embodiments, the system can use specific and targeted interventions for the specific patient. For example, the system may retrieve audio (e.g., a song that the resident likes, or a voice recording from a loved one of the resident) for the resident.

In at least one embodiment, the system can determine whether to select interventions based at least in part on whether the acuity score for the resident has changed (e.g., since the last score, in the last 24 hours, and the like), based on the magnitude of the change, based on the direction of the change, based on the value of the current score, and the like. For example, the acuity system may determine to generate an alert based on determining that the acuity score has increased, and the increase exceeds a threshold percentage and/or exceeds a threshold acuity value.

At block 1030, the system optionally implements the selected intervention(s). This may include a wide variety of actions, including revised resource allocations, outputting audio or other media, and the like.

### Example Method for Identifying Patient Attributes to Evaluate Acuity

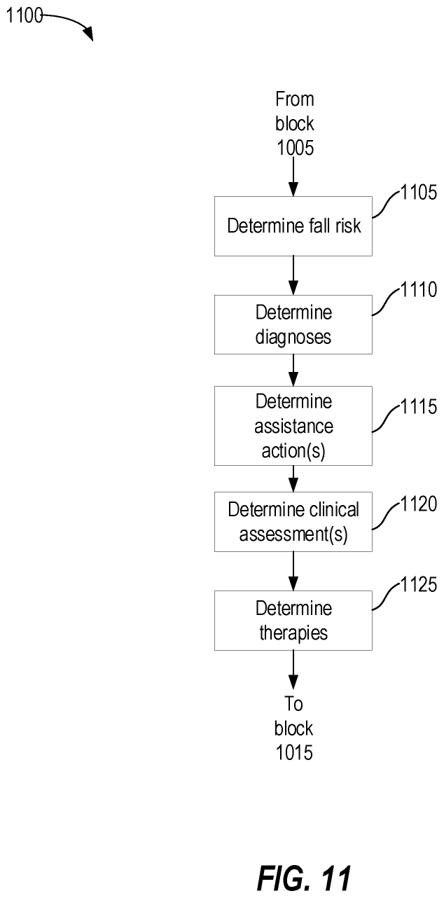

FIG. 11 is a flow diagram depicting an example method 1100 for identifying patient attributes to evaluate acuity. In some embodiments, the method 1100 is performed by an acuity system, such as the acuity system 140 of FIGS. 1 and 2. In some embodiments, the method 1100 provides additional detail for block 1010 of FIG. 10, and/or for block 915 of FIG. 9.

At block 1105, the acuity system can determine the fall risk of the resident. For example, as discussed above with reference to FIG. 3, the acuity system (or another system) may use one or more machine learning models (which may include one or more hybrid models) to predict the fall risk based on characteristics of the resident. In some embodiments, the fall risk includes multiple components, such as a fall probability, a predicted severity, and the like.

At block 1110, the acuity system can determine the diagnoses of the resident. For example, as discussed above with reference to FIG. 4, the acuity system may evaluate the resident data (e.g., searching based on defined ICD-10 or MDS codes) to determine whether the resident currently has any of the specified diagnoses.

At block 1115, the acuity system can determine the assistance actions needed by the resident. For example, as discussed above with reference to FIG. 5, the acuity system may evaluate the resident data to determine whether any of the specified assistances (e.g., ADLs, bathing assistance, and the like) are needed or requested by the resident.

At block 1120, the acuity system can determine the assessment(s) of one or more specified conditions of the resident. For example, as discussed above with reference to FIG. 6, the acuity system may search the resident data to determine whether any of the specified conditions have been indicated, the severity of each, and the like.

At block 1125, the acuity system can determine the therapies of the resident. For example, as discussed above with reference to FIG. 7, the acuity system may search the resident data to identify medications, interventions, or other therapies that the resident receives or has been prescribed. The method 1100 can then terminate, returning to block 1015 in FIG. 10 (to generate an acuity score for the resident based on the determined data), or to block 920 in FIG. 9 (to determine the historical acuity of the resident).

### Example Method for Generating and Aggregating Patient Acuity

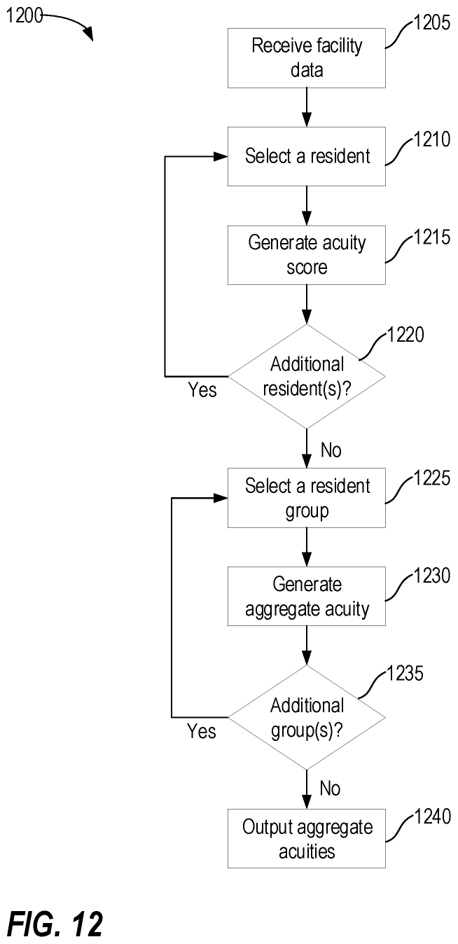

FIG. 12 is a flow diagram depicting an example method 1200 for generating and aggregating patient acuity. In some embodiments, the method 1200 is performed by an acuity system, such as the acuity system 140 of FIGS. 1 and 2.

At block 1205, the acuity system receives facility data. In an embodiment, the facility data can generally include data relating to the residents of a given facility. For example, in at least one aspect, the facility data can include, for each resident in the facility, a corresponding resident profiles (e.g., the resident data 205, in FIG. 2). As discussed above, this data can generally reflect attributes or characteristics of the residents, such as their diagnoses, assistance needs, and the like.

At block 1210, the acuity system selects one of the residents reflected in the facility data. Generally, this selection can be performed using any technique (including randomly or pseudo-randomly), as the acuity system will evaluate all of the facility's residents during the method 1200. Although an iterative or sequential process is depicted for conceptual clarity, in some embodiments, the acuity system may evaluate some or all of the resident data in parallel.

At block 1215, the acuity system generates an acuity score for the selected resident. For example, using the workflow 200 of FIG. 2 and/or the method 1000 of FIG. 10, the acuity system may process the attributes of the selected resident using the acuity model.

At block 1220, the acuity system determines whether there is at least one additional resident, in the facility, that has not-yet been evaluated. In some embodiments, this can include determining whether there are any residents with an out-of-date acuity score. That is, the acuity system can determine whether any of the remaining residents have updated data, as discussed above. If so, the method 1200 can return to block 1210. If an updated acuity score has been generated for all of the residents in the facility, the method 1200 continues to block 1225.

At block 1225, the acuity system selects a group of residents. In embodiments, these resident groups may be defined according to a wide variety of criteria, depending on the particular implementation. In at least one embodiment, the resident groups correspond to the physical distribution of residents in the facility. For example, the resident groups may correspond to separate wings, buildings, and the like. This can allow the acuity system to evaluate resident acuity on a per-region or per-locale basis, enabling improved resource allocation. In some embodiments, the resident groups are defined based on staffing assignments. For example, each resident group may correspond to a given caregiver (e.g., all residents primarily assigned to that caregiver), enabling the acuity system to evaluate resident acuity on a per-caregiver basis, which may help in balancing the workload of each caregiver and prevent or reduce mistakes or other negative outcomes caused by an overloaded caregiver.

At block 1230, the acuity system generates an aggregated acuity for the selected group. In some embodiments, the aggregated acuity can correspond to the average acuity of the group's members. In some embodiments, the aggregate acuity is the sum of the individual acuities of each resident in the group. In at least one embodiment, the aggregate acuity can indicate the distribution of acuities (e.g., based on classes or categories of acuity), as discussed above with reference to FIG. 8.

At block 1235, the acuity system determines whether there is at least one additional resident group that has not been evaluated. If so, the method 1200 returns to block 1225. If not, the method 1200 proceeds to block 1240.

At block 1240, the acuity system can output the aggregated acuities. For example, as discussed above with reference to FIG. 8, the acuity system may output the aggregate data on a GUI 800, enabling users to quickly review how acuity is distributed across the facility, across caregivers, and the like. In some embodiments, outputting the aggregate acuity can include outputting it to one or more downstream systems that can select and/or implement various interventions, as discussed above.

### Example Method for Processing Patient Attributes to Generate Acuity Scores

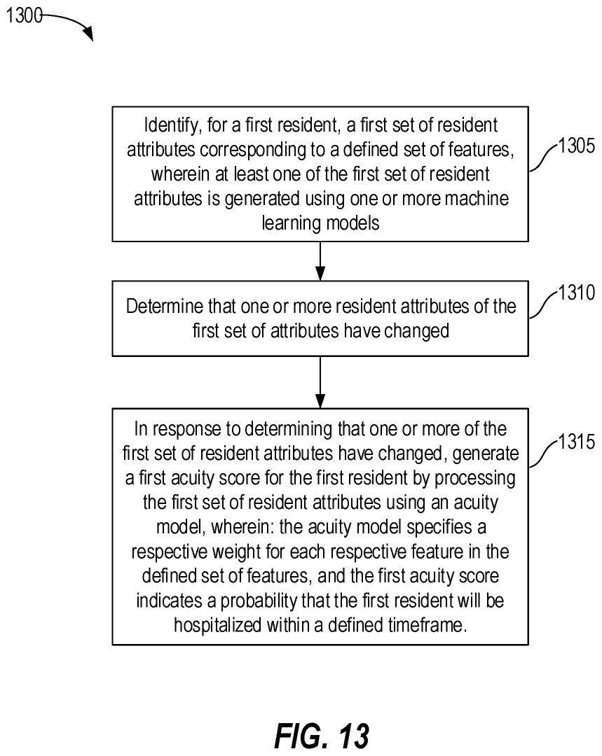

FIG. 13 is a flow diagram depicting an example method 1300 for generating acuity scores by processing patient attributes using an acuity model. In some embodiments, the method 1300 is performed by an acuity system, such as the acuity system 140 of FIGS. 1 and 2.

At block 1305, a first set of resident attributes (e.g., from resident data 205 from FIG. 2) corresponding to a defined set of features is identified, for a first resident, wherein at least one of the first set of resident attributes (e.g., fall risk data 215 of FIG. 2) is generated using one or more machine learning models.

At block 1310, it is determined that one or more resident attributes of the first set of attributes have changed.

At block 1315, in response to determining that one or more of the first set of resident attributes have changed, a first acuity score is generated for the first resident by processing the first set of resident attributes using an acuity model (e.g., the acuity model 145 of FIG. 1), wherein: the acuity model specifies a respective weight for each respective feature in the defined set of features, and the first acuity score indicates a probability that the first resident will be hospitalized within a defined timeframe.

### Example Method for Acuity Score Generation

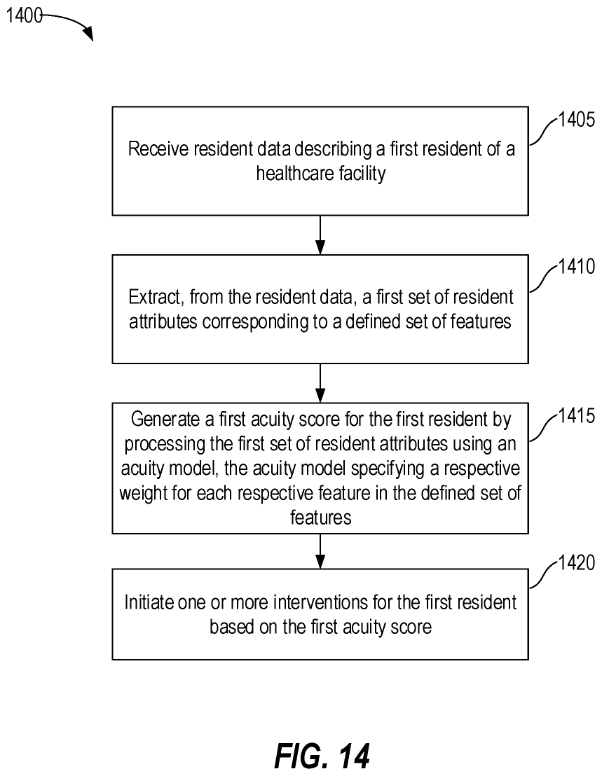

FIG. 14 is a flow diagram depicting an example method 1400 for generating an acuity score using an acuity model. In some embodiments, the method 1400 is performed by an acuity system, such as the acuity system 140 of FIGS. 1 and 2.

At block 1405, resident data (e.g., resident data 205 of FIG. 2) describing a first resident of a healthcare facility is received.

At block 1410, a first set of resident attributes corresponding to a defined set of features is extracted from the resident data.

At block 1415, a first acuity score (e.g., acuity score 235 of FIG. 2) is generated for the first resident by processing the first set of resident attributes using an acuity model (e.g., acuity model 145 of FIG. 1), the acuity model specifying a respective weight for each respective feature in the defined set of features.

At block 1420, one or more interventions (e.g., interventions 245 of FIG. 2) are initiated for the first resident based on the first acuity score.

### Example Processing System for Improved Acuity Modeling

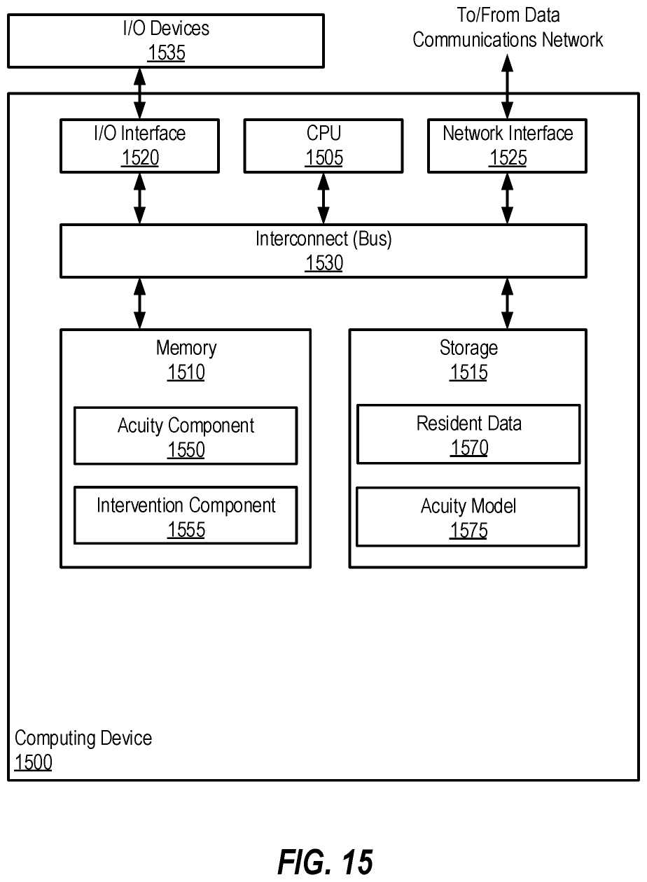

FIG. 15 depicts an example computing device 1500 configured to perform various aspects of the present disclosure. Although depicted as a physical device, in embodiments, the computing device 1500 may be implemented using virtual device(s), and/or across a number of devices (e.g., in a cloud environment). In one embodiment, the computing device 1500 corresponds to the acuity system 140 of FIG. 1 and FIG. 2.

As illustrated, the computing device 1500 includes a CPU 1505, memory 1510, storage 1515, a network interface 1525, and one or more I/O interfaces 1520. In the illustrated embodiment, the CPU 1505 retrieves and executes programming instructions stored in memory 1510, as well as stores and retrieves application data residing in storage 1515. The CPU 1505 is generally representative of a single CPU and/or GPU, multiple CPUs and/or GPUs, a single CPU and/or GPU having multiple processing cores, and the like. The memory 1510 is generally included to be representative of a random access memory. Storage 1515 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1535 (such as keyboards, monitors, etc.) are connected via the I/O interface(s) 1520. Further, via the network interface 1525, the computing device 1500 can be communicatively coupled with one or more other devices and components (e.g., via a network, which may include the Internet, local network(s), and the like). As illustrated, the CPU 1505, memory 1510, storage 1515, network interface(s) 1525, and I/O interface(s) 1520 are communicatively coupled by one or more buses 1530.

In the illustrated embodiment, the memory 1510 includes an acuity component 1550 and an intervention component 1555, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1510, in embodiments, the operations of the depicted components (and others not illustrated) may be implemented using hardware, software, or a combination of hardware and software.

In one embodiment, the acuity component 1550 is used to generate input data, to generate acuity models (e.g., based on historical data), and/or to generate acuity scores using acuity models, as discussed above. The intervention component 1555 may be configured to use the generated acuity scores to select, generate, and/or implement various interventions (such as alerts, prophylactic and specific/personalized interventions, and the like), as discussed above.

In the illustrated example, the storage 1515 includes resident data 1570 (which may correspond to historical data, such as historical data 105 of FIG. 1, and/or to current resident data, such as resident data 205 of FIG. 2, for one or more facilities), as well as an acuity model 1575. Although depicted as residing in storage 1515, the resident data 1570 and acuity model 1575 may be stored in any suitable location, including memory 1510.

### Example Clauses

Implementation examples are described in the following numbered clauses:

Clause 1: A method, comprising: identifying, for a first resident, a first set of resident attributes corresponding to a defined set of features, wherein at least one of the first set of resident attributes is generated using one or more machine learning models; determining that one or more resident attributes of the first set of attributes have changed; and in response to determining that one or more of the first set of resident attributes have changed, generating a first acuity score for the first resident by processing the first set of resident attributes using an acuity model, wherein: the acuity model specifies a respective weight for each respective feature in the defined set of features, and the first acuity score indicates a probability that the first resident will be hospitalized within a defined timeframe.

Clause 2: The method of Clause 1, further comprising: determining that the first acuity score exceeds a defined threshold; generating an alert identifying the first resident; selecting one or more interventions based on the first acuity score; and initiating the one or more interventions.

Clause 3: The method of any one of Clauses 1-2, wherein the respective weights are defined based on prior resident data for a plurality of residents, the prior resident data indicating, for each respective resident of the plurality of residents: a respective set of resident attributes, and a respective acuity score.

Clause 4: The method of any one of Clauses 1-3, further comprising: for each respective resident of a plurality of residents: identifying a respective set of resident attributes; and generating a respective acuity score for the respective resident by processing the respective set of resident attributes using the acuity model; and generating one or more aggregate acuity scores based on the respective acuity score of each respective resident of the plurality of residents.

Clause 5: A method, comprising: receiving resident data describing a first resident of a healthcare facility; extracting, from the resident data, a first set of resident attributes corresponding to a defined set of features; generating a first acuity score for the first resident by processing the first set of resident attributes using an acuity model, the acuity model specifying a respective weight for each respective feature in the defined set of features; and initiating one or more interventions for the first resident based on the first acuity score.

Clause 6: The method of Clause 5, further comprising: determining that the first acuity score exceeds a defined threshold; and generating an alert identifying the first resident.

Clause 7: The method of any one of Clauses 5-6, further comprising selecting the one or more interventions based on at least one of the first set of resident attributes.

Clause 8: The method of any one of Clauses 5-7, wherein the respective weights are defined based on prior resident data for a plurality of residents, the prior resident data indicating, for each respective resident of the plurality of residents: a respective set of resident attributes, and a respective acuity score.

Clause 9: The method of any one of Clauses 5-8, wherein the acuity model is a static statistical model with manually-curated weights.

Clause 10: The method of any one of Clauses 5-9, wherein the acuity model is a trained machine learning model, and wherein the respective weights were learned during a training phase.

Clause 11: The method of any one of Clauses 5-10, further comprising: for each respective resident of a plurality of residents in the healthcare facility: identifying a respective set of resident attributes; and generating a respective acuity score for the respective resident by processing the respective set of resident attributes using the acuity model.

Clause 12: The method of any one of Clauses 5-11, further comprising generating one or more aggregate acuity scores based on the respective acuity score of each respective resident of the plurality of residents.

Clause 13: The method of any one of Clauses 5-12, wherein: the first set of resident attributes comprises a predicted fall risk, and the predicted fall risk is generated by processing resident data using a trained machine learning model.

Clause 14: The method of any one of Clauses 5-13, wherein the defined set of features comprises: one or more features relating to resident diagnoses, one or more features relating to needed assistance actions, one or more features relating to clinical assessments, and one or more features relating to therapies.

Clause 15: The method of any one of Clauses 5-14, wherein: the one or more features relating to resident diagnoses comprise a defined set of diagnoses, and the first set of resident attributes indicate, for each respective diagnosis of the defined set of diagnoses, whether the first resident has the respective diagnosis.

Clause 16: The method of any one of Clauses 5-15, wherein the defined set of diagnoses comprises at least one of: (i) malnutrition, (ii) sarcopenia, (iii) congestive heart failure, (iv) chronic obstructive pulmonary disease (COPD), (v) cirrhosis, (vi) renal failure, (vii) chronic kidney disease, (viii) human immunodeficiency virus (HIV), (ix) diabetes, or (x) cancer.

Clause 17: The method of any one of Clauses 5-16, wherein: the one or more features relating to needed assistance actions comprise a defined set of actions that can be performed by one or more caregivers to assist residents, and the first set of resident attributes indicate, for each respective action of the defined set of actions, whether caregivers assist the first resident with the respective action.

Clause 18: The method of any one of Clauses 5-17, wherein the defined set of actions comprises at least one of: (i) assistance to eat, (ii) use a mechanical lift assist, (iii) bed mobility assistance, (iv) transfer assistance, (v) walking assistance, or (vi) bathing assistance.

Clause 19: The method of any one of Clauses 5-18, wherein: the one or more features relating to clinical assessments comprise a defined set of conditions, recorded by one or more caregivers, relating to functional states of residents, and the first set of resident attributes indicate, for each respective condition of the defined set of conditions, whether the first resident has the respective condition.

Clause 20: The method of any one of Clauses 5-19, wherein the defined set of conditions comprises at least one of: (i) weight loss, (ii) use of intravenous (IV) feeding, (iii) congestive heart failure, (iv) pain, (v) incontinence, (vi) a catheter or ostomy, (vii) a deep tissue injury, (viii) a skin tear, (ix) an ulcer, (x) use of supplemental oxygen, (xi) use of a bilevel positive airway pressure (BIPAP) device, (xii) required isolation, (xiii) one or more mood or behavioral issues, or (xiv) one or more hallucinations or delusions.

Clause 21: The method of any one of Clauses 5-20, wherein: the one or more features relating to therapies comprise a defined set of therapies, and the first set of resident attributes indicate, for each respective therapy of the defined set of therapies, whether the first resident receives the respective therapy.

Clause 22: The method of any one of Clauses 5-21, wherein the defined set of therapies comprises at least one of: (i) total parenteral nutrition, (ii) psychotropic medication, (iii) anticoagulant medication, or (iv) blood glucose monitoring.

Clause 23: A system, comprising: a memory comprising computer-executable instructions; and one or more processors configured to execute the computer-executable instructions and cause the processing system to perform a method in accordance with any one of Clauses 1-22.

Clause 24: A system, comprising means for performing a method in accordance with any one of Clauses 1-22.

Clause 25: A non-transitory computer-readable medium comprising computer-executable instructions that, when executed by one or more processors of a processing system, cause the processing system to perform a method in accordance with any one of Clauses 1-22.

Clause 26: A computer program product embodied on a computer-readable storage medium comprising code for performing a method in accordance with any one of Clauses 1-22.

### ADDITIONAL CONSIDERATIONS

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., the acuity system 140) or related data available in the cloud. For example, the acuity system 140 could execute on a computing system in the cloud and generate and/or use acuity models. In such a case, the acuity system 140 could generate models to evaluate medical acuity, and store the models at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
identifying, for a first resident, a first set of resident attributes corresponding to a defined set of features, wherein at least one of the first set of resident attributes is generated using one or more machine learning models;
determining that one or more resident attributes of the first set of attributes have changed; and
in response to determining that one or more of the first set of resident attributes have changed, generating a first acuity score for the first resident by processing the first set of resident attributes using an acuity model, wherein:
the acuity model specifies a respective weight for each respective feature in the defined set of features, and
the first acuity score indicates a probability that the first resident will be hospitalized within a defined timeframe.

2. The method of claim 1, further comprising:
determining that the first acuity score exceeds a defined threshold;
generating an alert identifying the first resident;
selecting one or more interventions based on the first acuity score; and
initiating the one or more interventions.

3. The method of claim 1, wherein the respective weights are defined based on prior resident data for a plurality of residents, the prior resident data indicating, for each respective resident of the plurality of residents:
a respective set of resident attributes, and
a respective acuity score.

4. The method of claim 1, further comprising:
for each respective resident of a plurality of residents:
identifying a respective set of resident attributes; and
generating a respective acuity score for the respective resident by processing the respective set of resident attributes using the acuity model; and
generating one or more aggregate acuity scores based on the respective acuity score of each respective resident of the plurality of residents.

5. A method, comprising:
receiving resident data describing a first resident of a healthcare facility;
extracting, from the resident data, a first set of resident attributes corresponding to a defined set of features;
generating a first acuity score for the first resident by processing the first set of resident attributes using an acuity model, the acuity model specifying a respective weight for each respective feature in the defined set of features; and
initiating one or more interventions for the first resident based on the first acuity score.

6. The method of claim 5, further comprising:
determining that the first acuity score exceeds a defined threshold; and
generating an alert identifying the first resident.

7. The method of claim 6, further comprising selecting the one or more interventions based on at least one of the first set of resident attributes.

8. The method of claim 5, wherein the respective weights are defined based on prior resident data for a plurality of residents, the prior resident data indicating, for each respective resident of the plurality of residents:
a respective set of resident attributes, and
a respective acuity score.

9. The method of claim 8, wherein the acuity model is a static statistical model with manually-curated weights.

10. The method of claim 8, wherein the acuity model is a trained machine learning model, and wherein the respective weights were learned during a training phase.

11. The method of claim 5, further comprising:
for each respective resident of a plurality of residents in the healthcare facility:
identifying a respective set of resident attributes; and
generating a respective acuity score for the respective resident by processing the respective set of resident attributes using the acuity model.

12. The method of claim 11, further comprising generating one or more aggregate acuity scores based on the respective acuity score of each respective resident of the plurality of residents.

13. The method of claim 5, wherein:
the first set of resident attributes comprises a predicted fall risk, and
the predicted fall risk is generated by processing resident data using a trained machine learning model.

14. The method of claim 5, wherein the defined set of features comprises:
one or more features relating to resident diagnoses,
one or more features relating to needed assistance actions,
one or more features relating to clinical assessments, and
one or more features relating to therapies.

15. The method of claim 14, wherein:
the one or more features relating to resident diagnoses comprise a defined set of diagnoses, and
the first set of resident attributes indicate, for each respective diagnosis of the defined set of diagnoses, whether the first resident has the respective diagnosis.

16. The method of claim 15, wherein the defined set of diagnoses comprises at least one of: (i) malnutrition, (ii) sarcopenia, (iii) congestive heart failure, (iv) chronic obstructive pulmonary disease (COPD), (v) cirrhosis, (vi) renal failure, (vii) chronic kidney disease, (viii) human immunodeficiency virus (HIV), (ix) diabetes, or (x) cancer.

17. The method of claim 14, wherein:
the one or more features relating to needed assistance actions comprise a defined set of actions that can be performed by one or more caregivers to assist residents, and
the first set of resident attributes indicate, for each respective action of the defined set of actions, whether caregivers assist the first resident with the respective action.

18. The method of claim 17, wherein the defined set of actions comprises at least one of: (i) assistance to eat, (ii) use a mechanical lift assist, (iii) bed mobility assistance, (iv) transfer assistance, (v) walking assistance, or (vi) bathing assistance.

19. The method of claim 14, wherein:
the one or more features relating to clinical assessments comprise a defined set of conditions, recorded by one or more caregivers, relating to functional states of residents, and
the first set of resident attributes indicate, for each respective condition of the defined set of conditions, whether the first resident has the respective condition.

20. The method of claim 19, wherein the defined set of conditions comprises at least one of: (i) weight loss, (ii) use of intravenous (IV) feeding, (iii) congestive heart failure, (iv) pain, (v) incontinence, (vi) a catheter or ostomy, (vii) a deep tissue injury, (viii) a skin tear, (ix) an ulcer, (x) use of supplemental oxygen, (xi) use of a bilevel positive airway pressure (BIPAP) device, (xii) required isolation, (xiii) one or more mood or behavioral issues, or (xiv) one or more hallucinations or delusions.

21. The method of claim 14, wherein:
the one or more features relating to therapies comprise a defined set of therapies, and
the first set of resident attributes indicate, for each respective therapy of the defined set of therapies, whether the first resident receives the respective therapy.

22. The method of claim 21, wherein the defined set of therapies comprises at least one of: (i) total parenteral nutrition, (ii) psychotropic medication, (iii) anticoagulant medication, or (iv) blood glucose monitoring.

23. A method, comprising:
receiving resident data describing a first resident of a healthcare facility;
extracting, from the resident data, a first set of resident attributes corresponding to a defined set of features;
determining a first acuity score for the first resident based on the first set of resident attributes;
training an acuity model based on the first acuity score and the first set of resident attributes; and
deploying the acuity model.

24. The method of claim 23, wherein training the acuity model comprises generating a respective weight for each respective feature in the defined set of features based on the resident data and based further on prior resident data for a plurality of residents, the prior resident data indicating, for each respective resident of the plurality of residents:
a respective set of resident attributes, and
a respective acuity score.

25. The method of claim 23, wherein:
the first set of resident attributes comprises a predicted fall risk, and
the predicted fall risk is generated by processing resident data using a trained machine learning model.

26. The method of claim 23, wherein the defined set of features comprises:
one or more features relating to resident diagnoses,
one or more features relating to needed assistance actions,
one or more features relating to clinical assessments, and
one or more features relating to therapies.

27. The method of claim 26, wherein:
the one or more features relating to resident diagnoses comprise a defined set of diagnoses, and
the first set of resident attributes indicate, for each respective diagnosis of the defined set of diagnoses, whether the first resident has the respective diagnosis.

28. The method of claim 27, wherein the defined set of diagnoses comprises at least one of: (i) malnutrition, (ii) sarcopenia, (iii) congestive heart failure, (iv) chronic obstructive pulmonary disease (COPD), (v) cirrhosis, (vi) renal failure, (vii) chronic kidney disease, (viii) human immunodeficiency virus (HIV), (ix) diabetes, or (x) cancer.

29. The method of claim 26, wherein:
the one or more features relating to needed assistance actions comprise a defined set of actions that can be performed by one or more caregivers to assist residents, and
the first set of resident attributes indicate, for each respective action of the defined set of actions, whether caregivers assist the first resident with the respective action.

30. The method of claim 29, wherein the defined set of actions comprises at least one of: (i) assistance to eat, (ii) use a mechanical lift assist, (iii) bed mobility assistance, (iv) transfer assistance, (v) walking assistance, or (vi) bathing assistance.

31. The method of claim 26, wherein:
the one or more features relating to clinical assessments comprise a defined set of conditions, recorded by one or more caregivers, relating to functional states of residents, and
the first set of resident attributes indicate, for each respective condition of the defined set of conditions, whether the first resident has the respective condition.

32. The method of claim 31, wherein the defined set of conditions comprises at least one of: (i) weight loss, (ii) use of intravenous (IV) feeding, (iii) congestive heart failure, (iv) pain, (v) incontinence, (vi) a catheter or ostomy, (vii) a deep tissue injury, (viii) a skin tear, (ix) an ulcer, (x) use of supplemental oxygen, (xi) use of a bilevel positive airway pressure (BIPAP) device, (xii) required isolation, (xiii) one or more mood or behavioral issues, or (xiv) one or more hallucinations or delusions.

33. The method of claim 26, wherein:
the one or more features relating to therapies comprise a defined set of therapies, and
the first set of resident attributes indicate, for each respective therapy of the defined set of therapies, whether the first resident receives the respective therapy.

34. The method of claim 33, wherein the defined set of therapies comprises at least one of: (i) total parenteral nutrition, (ii) psychotropic medication, (iii) anticoagulant medication, or (iv) blood glucose monitoring.

35. A system, comprising:
one or more computer processors; and
logic encoded in a non-transitory medium, the logic executable by operation of the one or more computer processors to perform an operation comprising:
receiving resident data describing a first resident of a healthcare facility;
extracting, from the resident data, a first set of resident attributes corresponding to a defined set of features;
generating a first acuity score for the first resident by processing the first set of resident attributes using an acuity model, the acuity model specifying a respective weight for each respective feature in the defined set of features; and
initiating one or more interventions for the first resident based on the first acuity score.
